(19) United States
(12) Patent Application Publication
(10) Pub. No.: US 2020/0316037 A1
(43) Pub. Date: Oct. 8, 2020

(54) COMPOSITIONS AND METHODS FOR TREATING NEOPLASIA

(71) Applicant: THE ANGIOGENESIS FOUNDATION, Cambridge, MA (US)

(72) Inventors: Vincent W. LI, Holliston, MA (US); William W. LI, Holliston, MA (US)

(73) Assignee: THE ANGIOGENESIS FOUNDATION, Cambridge, MA (US)

(21) Appl. No.: 16/956,740

(22) PCT Filed: Dec. 21, 2018
(86) PCT No.: PCT/US2018/067283

§ 371 Date: Jun. 22, 2020

Related U.S. Application Data
(60) Provisional application No. 62/609,109, filed on Dec. 21, 2017.

(51) Int. Cl. A61K 31/437; A61K 31/196; A61K 31/415; A61K 31/573; A61K 31/203; A61K 31/047; A61K 31/436; A61K 47/40; A61K 9/00; A61K 47/36; A61K 47/38; A61K 47/42; A61P 35/00

(52) U.S. Cl. CPC A61K 31/437; A61K 31/196; A61K 31/415; A61K 31/573; A61K 31/203; A61K 31/047; A61P 35/00; A61K 47/40; A61K 9/0014; A61K 9/0053; A61K 47/36; A61K 47/38; A61K 47/42; A61K 31/436

(57) ABSTRACT

The invention relates to unique compositions and methods for treating neoplasia. Specifically, the invention relates to an off-label combinatorial therapy that modulates angiogenesis and immune response to treat neoplasia.

### CROSS-REFERENCE TO RELATED APPLICATIONS

This application claims priority to and the benefit of U.S. Provisional Patent Application 62/609,109, filed Dec. 21, 2017, which is incorporated by reference herein in its entirety.

### FIELD OF THE INVENTION

The invention relates to compositions and methods for treating a neoplasia. Specifically, the invention relates to an off-label combinatorial therapy for treating a neoplasia.

### BACKGROUND OF THE INVENTION

Several large patient populations are diagnosed with malignant and benign neoplasia annually, and about half a million people in the United States die of malignant neoplasia annually. Although medical improvements in neoplasia detection, diagnosis, and treatment have increased the survival rate for many types of such neoplasia, only about 60 percent of patients diagnosed with such neoplasia are alive five years after treatment, making such neoplasia the second leading cause of death in the United States.

Nonmelanoma skin cancer is an important neoplasia. Approximately 5.4 million patients in the U.S. are diagnosed annually with nonmelanoma skin cancer, including basal cell carcinoma (BCC), squamous cell carcinoma in situ (SCCIS) and squamous cell carcinoma (SCC). Three times more nonmelanoma skin cancers develop each year than cancers of all other types combined. In addition, the prevalence of skin cancers is growing, doubling in the past 50 years. Nearly half of people over age 65 will develop one form of nonmelanoma skin cancer. In immunosuppressed patients such as people on systemic steroids on biologic therapies, or with organ transplants, the risk can be 100-fold higher. In cases of extensive disease or severe comorbidities, conventional treatment modalities such as surgical excision (FIG. 1A) or topical chemotherapy, cryotherapy, or radiation therapy (FIG. 1B) may be impractical, physically disfiguring, painful, technically unfeasible, unavailable in remote or underdeveloped areas, and may impair quality of life. Another limitation of conventional treatment approaches is incomplete eradication of the cancer resulting in recurrence as high as 10% for certain modalities. Because cancers likely form over a larger area than the visible lesion (e.g. “field cancerization”), local surgery often does not completely eliminate microscopic atypical cells.

A multitude of factors interact with one another to promote tumorigenesis and tumor survival. One such factor is aberrant angiogenesis, which promotes tumor progression. Targeting the blood vessels that feed tumors is an opportunity to intercept early cancers, treat established tumors, and prevent metastatic spread.

Because angiogenesis is a critical event, an antiangiogenic therapy can be a molecular strategy for treating a neoplasia such as a nonmelanoma skin cancer and other malignant cancers.

Because angiogenesis is also a critical event in the genesis and growth of benign neoplasias, an antiangiogenic therapy can be a molecular strategy for treating a benign neoplasia such as an adenoma, fibroma or hemangioma.

Other critical events in the development or progression of neoplasia include inflammation; an immunologically-suppressive milieu within the tumor microenvironment; unchecked cell proliferation, and abnormal cell maturation. Therefore, therapies which suppress inflammation or stimulate the innate immune system or suppress immune evasion by cancer cells, or inhibit proliferation or promote normal cell maturation can be effective strategies to treat neoplasia.

A wealth of valuable experience already exists in FDA-approved drugs for other conditions, unrelated to cancer. Many such FDA approved drugs are off-patent, providing opportunities to productize novel combinations. Combinations of drugs that target different and multiple pathways in cancer or address elements in the cancer microenvironment offer the best chance at eradicating disease.

Accordingly, there exists a need for improved compositions and methods for providing an antiangiogenic and immunotherapeutic solution to treat various neoplasia including nonmelanoma skin cancers, other malignant cancers and benign neoplasias.

### SUMMARY OF THE INVENTION

In one aspect, the invention provides a composition comprising: a toll-like receptor 7 (TLR7) agonist, non-steroidal anti-inflammatory drug(s) (NSAIDs), a glucocorticoid anti-inflammatory agent, a vitamin A derivative, a vitamin D3 derivative, a mammalian target of rapamycin (mTOR) inhibitor or a combination thereof, and pharmaceutically acceptable carriers. In an exemplary embodiment, said TLR7 agonist is imiquimod; said NSAID is diclofenac, or celecoxib, or a combination thereof; said glucocorticoid anti-inflammatory agent is hydrocortisone valerate; said vitamin A derivative is tretinoin; said vitamin D3 derivative is calcipotriene; and said mTOR inhibitor is sirolimus.

In another aspect, the invention provides a method of treating a neoplasia in a subject, the method comprising: administering to said subject a therapeutically effective amount of a toll-like receptor 7 (TLR7) agonist, a non-steroidal anti-inflammatory drug (NSAID), a glucocorticoid anti-inflammatory agent, a vitamin A derivative, a vitamin D3 derivative, a mechanistic target of rapamycin (mTOR) inhibitor, or a combination thereof, thereby treating said neoplasia in said subject.

In another aspect, the invention provides a method of targeting angiogenesis to treat a neoplasia in a subject, the method comprising: administering to said subject a therapeutically effective amount of a toll-like receptor 7 (TLR7) agonist, a non-steroidal anti-inflammatory drug (NSAID), a glucocorticoid anti-inflammatory agent, a vitamin A derivative, a vitamin D3 derivative, a mechanistic target of rapamycin (mTOR) inhibitor, or a combination thereof, thereby targeting said tumor angiogenesis to treat said neoplasia in said subject.

In another aspect, the invention provides a method of modulating the immune system to treat a neoplasia in a subject, the method comprising: administering to said subject a therapeutically effective amount of a toll-like receptor 7 (TLR7) agonist, a non-steroidal anti-inflammatory drug (NSAID), a glucocorticoid anti-inflammatory agent, a vitamin A derivative, a vitamin D3 derivative, a mechanistic target of rapamycin (mTOR) inhibitor, or a combination thereof, thereby targeting said tumor angiogenesis and improving immune-directed cancer cell targeting to treat said neoplasia in said subject.

In another aspect, the invention provides a method of targeting proliferative cells to treat a neoplasia in a subject, the method comprising: administering to said subject a therapeutically effective amount of a toll-like receptor 7 (TLR7) agonist, a non-steroidal anti-inflammatory drug (NSAID), a glucocorticoid anti-inflammatory agent, a vitamin A derivative, a vitamin D3 derivative, a mechanistic target of rapamycin (mTOR) inhibitor, or a combination thereof, thereby targeting said tumor angiogenesis and improving immune-directed cancer cell targeting to treat said neoplasia in said subject.

In another aspect, the invention provides a method of promoting normal cell maturation to treat a neoplasia in a subject, the method comprising: administering to said subject a therapeutically effective amount of a toll-like receptor 7 (TLR7) agonist, a non-steroidal anti-inflammatory drug (NSAID), a glucocorticoid anti-inflammatory agent, a vitamin A derivative, a vitamin D3 derivative, a mechanistic target of rapamycin (mTOR) inhibitor, or a combination thereof, thereby targeting said tumor angiogenesis and improving immune-directed cancer cell targeting to treat said neoplasia in said subject.

In another aspect, the invention provides a method of specifically treating a nonmelanoma skin cancer in a subject, the method comprising: administering to said subject a therapeutically effective amount of a toll-like receptor 7 (TLR7) agonist, a non-steroidal anti-inflammatory drug (NSAID), a glucocorticoid anti-inflammatory agent, a vitamin A derivative, a vitamin D3 derivative, a mechanistic target of rapamycin (mTOR) inhibitor, or a combination thereof, thereby treating said nonmelanoma skin cancer in said subject.

In another aspect, the invention provides a method of treating multiple forms of melanoma skin cancer in a subject, the method comprising: administering to said subject a therapeutically effective amount of a toll-like receptor 7 (TLR7) agonist, a non-steroidal anti-inflammatory drug (NSAID), a glucocorticoid anti-inflammatory agent, a vitamin A derivative, a vitamin D3 derivative, a mechanistic target of rapamycin (mTOR) inhibitor, or a combination thereof, thereby treating said melanoma skin cancer in said subject.

In another aspect, the invention provides a method of providing a neoadjuvant therapy for treating a neoplasia. The neoadjuvant therapy can be provided using the compositions described herein, prior to another form of therapy in order to make the other form of therapy more effective or diminishing the consequences of the other form of therapy. In some embodiments, the neoadjuvant therapy can be provided using combinations of therapies that were not originally intended for treating such neoplasia or not originally intended for topical method of application. Therefore, the usage of such combination therapies in this invention are off label, in that their usage differs from the package label that specifies treatment for a specific disease and in a manner. An example would be to use this invention prior to surgery in order to reduce the excision size of the surgery and reduce the subsequent scar.

In another aspect, the invention provides a method of treating neoplasia using combinations of therapies that were not originally intended for treating such neoplasia or not originally intended for topical method of application. Therefore, the usage of such combination therapies in this invention are off label, in that their usage differs from the package label that specifies treatment for a specific disease and in a manner.

In another aspect, the invention provides a method of treating neoplasia using dosages of therapies that are significantly below therapeutically effective doses for the conditions the therapies in isolation (as monotherapy) were originally designed to treat.

In another aspect, the invention provides a method of treating neoplasia by adjusting dose frequencies of therapies at an individual user level that avoids undesirable local inflammatory reactions and other undesirable side effects.

In another aspect, the invention provides a method of determining the optimal dose through remote transmission of visual images and response to survey of symptoms.

In another aspect, the invention provides a method of combining agents through mechanical means either at the time of application using a kit or prepared in advance.

In another aspect, the invention provides a method of defining an appropriate treatment quantity through metered applicator.

In another aspect, the invention provides a means of treating field cancerization of skin over a broad area, not amenable to conventional therapies that target a discrete area.

In another aspect, the invention provides a method of diagnosing microscopic neoplasia not yet visible to the eye by the appearance of a visible mild local tissue response.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTIONS OF THE DRAWINGS

A brief summary of each of the figures described in this specification are provided below. This application contains at least one figure executed in color. Copies of this application with color drawings will be provided upon request and payment of the necessary fee.

FIGS. 1A and 1B show the pictures of disfigurement and morbidity after conventional treatments for treating nonmelanoma skin cancer. FIG. 1 A shows disfigurement after surgical treatment and FIG. 1B shows disfigurations after topical chemotherapy, cryotherapy, and radiation therapy.

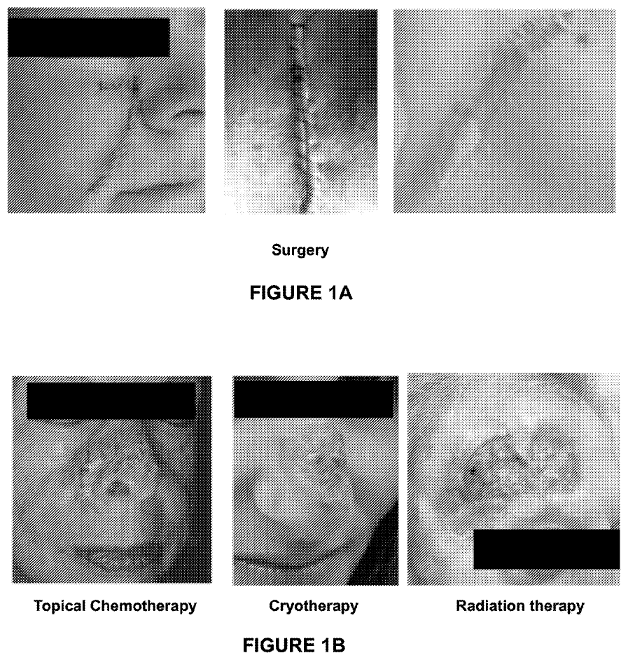

FIG. 2 shows the role of angiogenesis in skin cancers. BCC refers to basal cell carcinoma and SCC refers to squamous cell carcinoma.

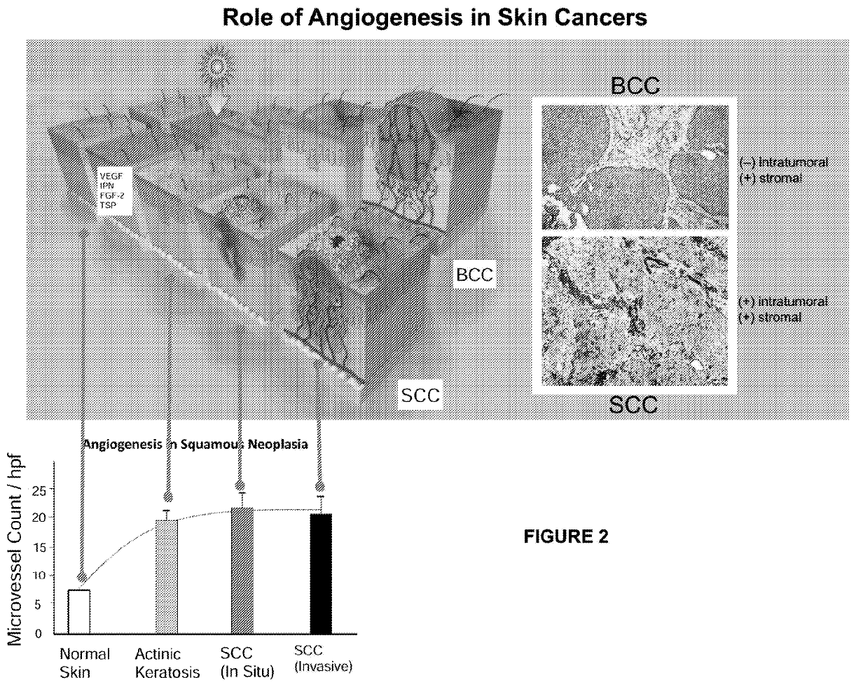

FIG. 3 shows treatment of cancers by targeting angiogenesis.

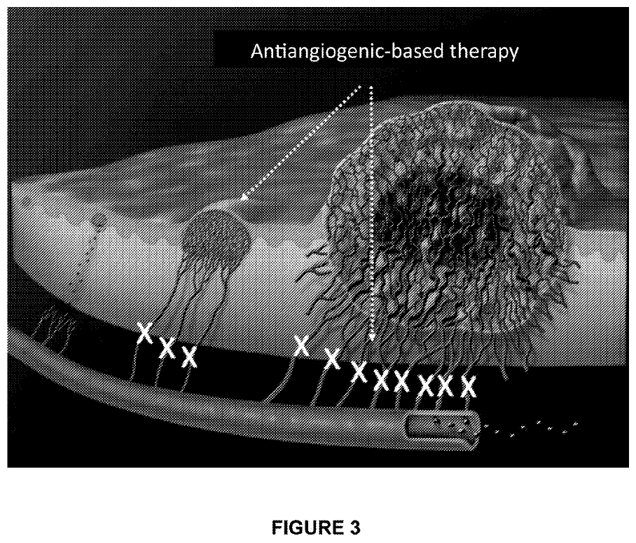

FIG. 4 shows the chemical structures of Composition I active ingredients.

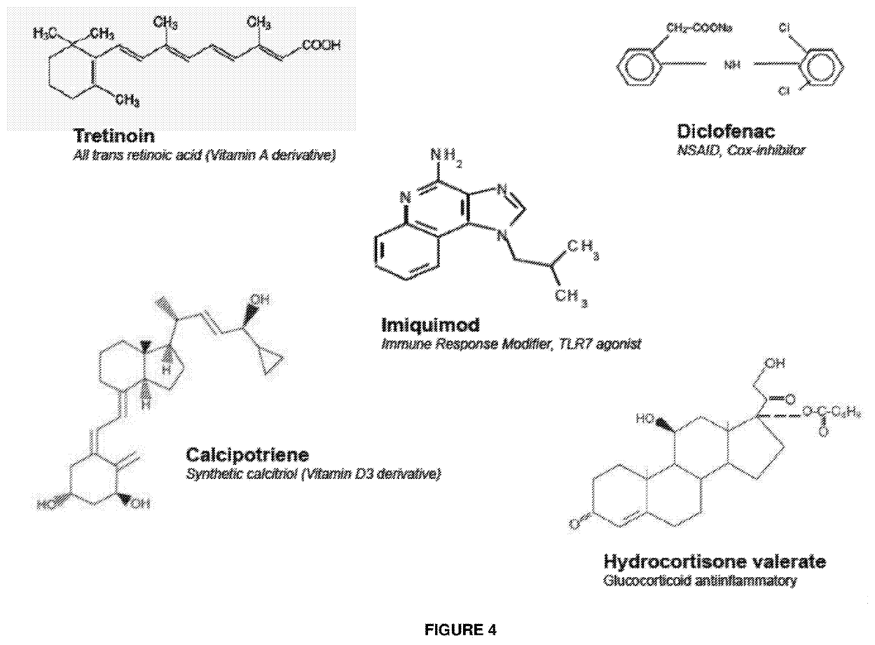

FIG. 5 shows the chemical structures of active ingredients for the Composition II related therapies.

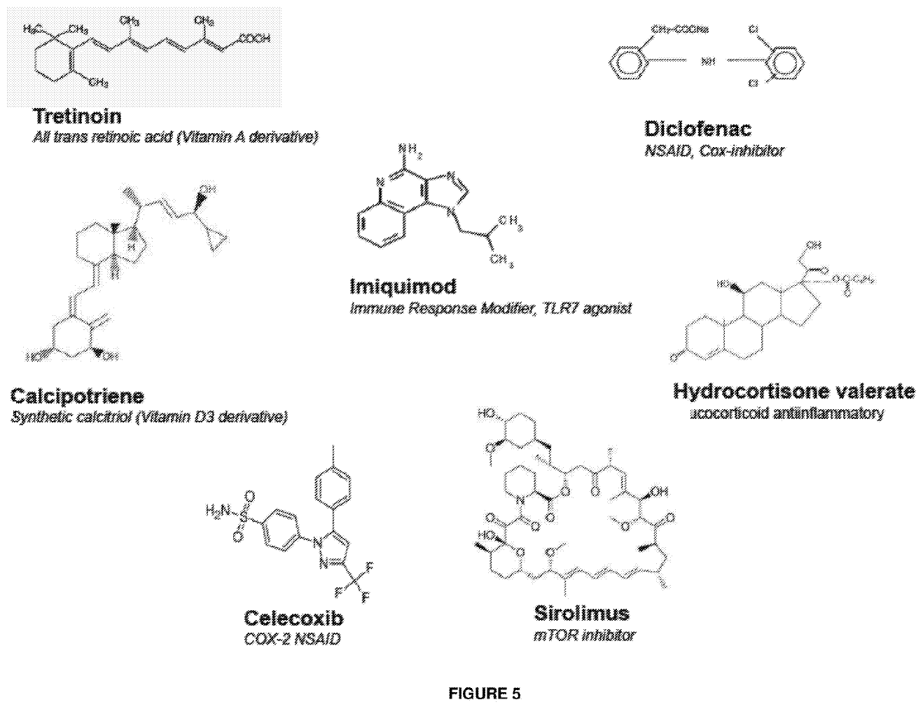

FIG. 6 shows a table of Composition I and II components and their FDA approved indications.

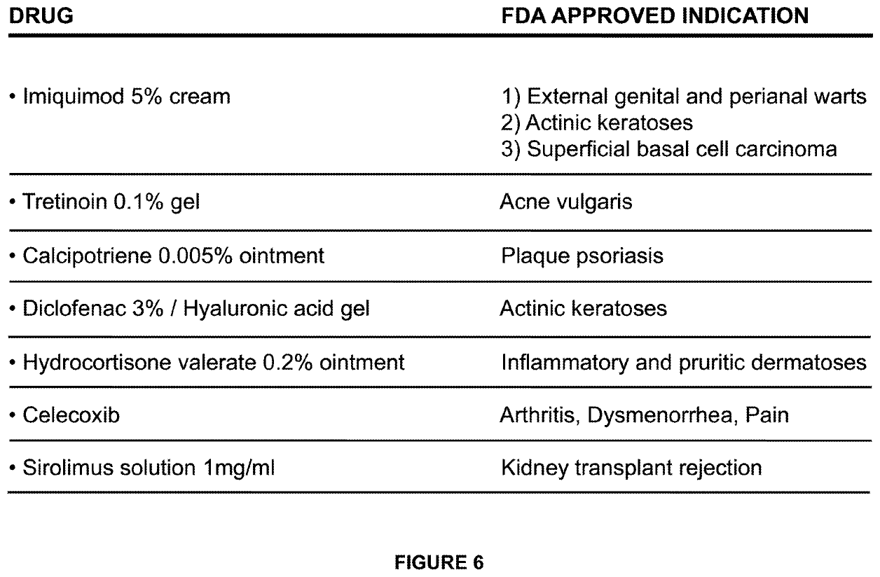

FIG. 7 shows a table of Composition I and II components and their angiogenesis targets.

FIGS. 8A and 8B show the treatment of a basal cell carcinoma with the topical use of Composition I. FIG. 8A shows the skin before treatment and FIG. 8B shows the skin after treatment.

FIG. 9 shows the treatment of numerous basal cell carcinomas (>40) with the topical use of Composition I. Two pictures on the left show before treatment and the picture on the right shows after treatment.

FIGS. 10A and 10B show the treatment of a basal cell carcinoma with the topical use of Composition II. FIG. 10A shows the skin before treatment and FIG. 10B shows the skin after treatment.

FIG. 11 shows the treatment of a basal cell carcinoma with the topical use of with the topical use of Composition III. The treatment effects monitored on days 0, 6, 10, 13, 16, and 17 show rapid regression of the cancer. Topical use of Composition II is rapidly effective in treating a basal cell carcinoma.

FIG. 12 shows topical treatment with the use of Composition I of a recurrent, invasive squamous cell carcinoma which recurred despite conventional surgical treatment. Bottom left picture shows cancer arising within the surgical scar. Bottom right shows clearance after topical treatment, confirmed by post-treatment biopsy.

FIG. 13 shows treatment of an invasive squamous cell carcinoma with the use of Composition I. Top pictures show the clinical and pathological images before treatment. Bottom pictures show after treatment.

FIG. 14 shows treatment of an invasive squamous cell carcinoma with the use of Composition III. Top picture and bottom left picture shows the tumor before treatment. Bottom middle picture shows significant interval improvement after 1 month of treatment. Bottom right picture shows clearance after 3 months of topical treatment.

FIG. 15 shows a summary table of clinical results for treating basal cell carcinoma with the use of Composition I.

FIG. 16 shows a summary table of clinical results for treating squamous cell carcinoma in situ with the use of Composition I.

FIG. 17 shows a summary table of clinical results for treating invasive squamous cell carcinoma with the use of Composition I.

FIG. 18 shows a table of clinical results for treating skin cancers (basal cell carcinoma (BCC); squamous cell carcinoma in situ (SCCIS); and invasive squamous cell carcinoma (SCC)) with the use of Composition II.

FIG. 19 shows a table of clinical results for treating skin cancers (basal cell carcinoma (BCC); squamous cell carcinoma in situ (SCCIS); and invasive squamous cell carcinoma (SCC)) with the use of Composition III.

FIG. 20 shows novel dosing scheme. Any suitable dosing algorithm, known to one of skilled in the art, can be used. Algorithm can enable tailoring of administration of Composition I or II or III per individual patient. Therapeutic efficacy can be achieved without undesirable irritation and inflammation using the combination of agents. Dose frequency can be increased to reach tissue reaction, but not undesirable clinical inflammation. Algorithm, known in the art, can allow for optimizing biological effects and minimizing undesirable tissue side effects (gross inflammation). Expected reaction may include mild-moderate erythema (vasodilation), minimal purpura, and mild local swelling. Undesired reaction includes inflammation, itching, stinging, pain, erosion, and local bleeding.

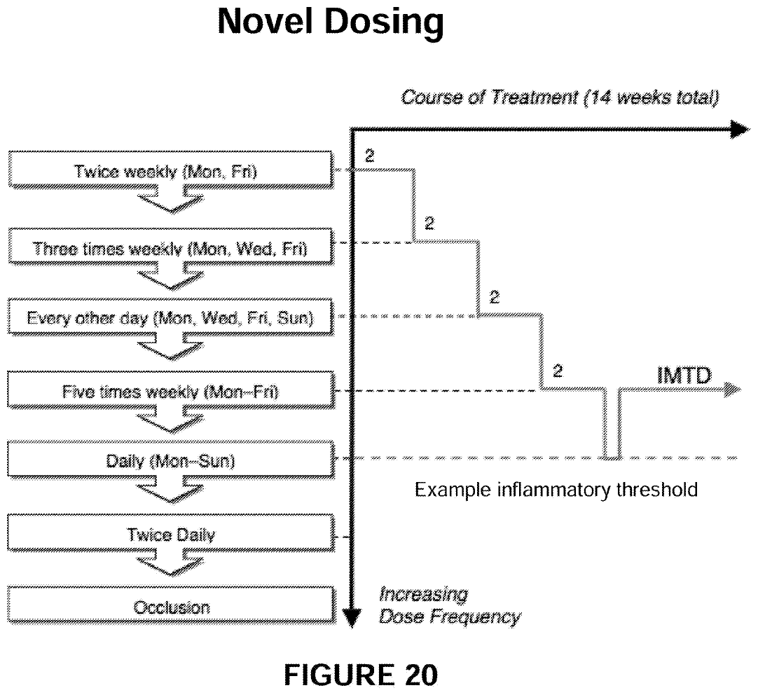

FIG. 21 shows the percentage of individuals at each dose frequency to who achieved optimal outcomes (complete clearance of cancer without any undesired local side effects) in the topical treatment of non-melanoma skin cancer. All patients were effectively cleared of their skin cancers without any discomfort or gross inflammation. The frequency of treatment was titrated and final dose frequency determined by individualized tissue response. A more personalized approach to treatment was accomplished.

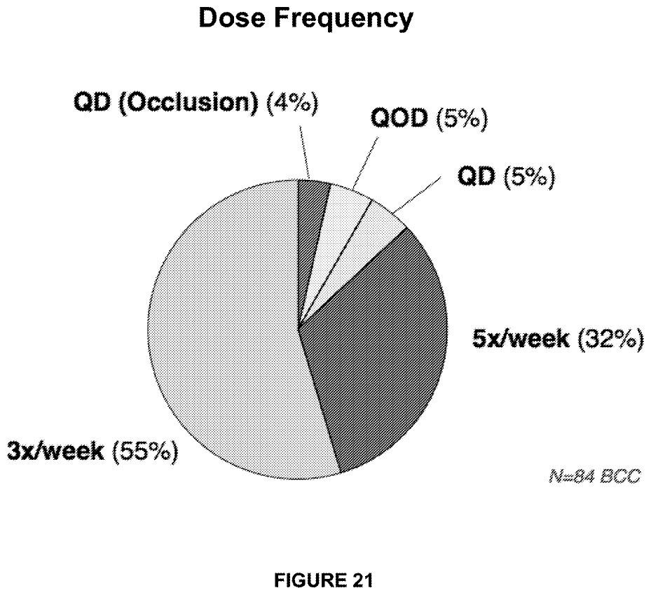

FIG. 22 shows the antiangiogenic effects of the combinatorial composition on blood vessels resulting in reduced microvessel density (MVD). MVD was measured by staining the tissue sample for CD31 and counting hotspots. There was increased MVD in SCC tumor stroma compared to normal skin controls. Antiangiogenic treatment with Composition I showed decreased vessels density (Pre-treatment average MVD=36.0 versus Post-treatment average MVD=19.8).

FIG. 23 shows normalization of abnormal vessels after treatment with Composition I, as evidenced by vessel maturation (expression of alpha smooth muscle actin) and pruning of microvessels (highlighted by CD31 staining).

FIG. 24 shows quality of life as quantified by users during treatment with Composition I. Patients were queried about their quality of life while on Composition I compared to previous therapies (“5”=no impairment, “0”=major impairment compromising work or normal activities). Treatment with the combinatorial composition exhibited the highest quality of life compared to conventional therapies such as surgery electrodessication and curettage (EDC) and cryotherapy.

FIG. 25 shows self-rated cosmetic outcomes after treatment with Composition I. Treatment with the combinatorial composition exhibited the best cosmetic outcome relative to other treatments, based on user's self-perception. Patients were queried about their cosmetic outcome after successful completion of Composition I compared to previous therapies conventional (“5”=scarless, “0”=significant disfigurement). Excellent cosmetic outcomes were achieved without contour irregularity, atrophy, hypertrophic scar, or depigmentation.

FIG. 26 shows treatment of an oral squamous cell carcinoma with Composition I. Top left shows before treatment. Top right shows after treatment. Bottom picture shows post-treatment biopsy which was reported as dramatic improvement in papillary proliferation, with overall preservation of the basal layer integrity.

FIG. 27 shows treatment of an angiosarcoma with Composition I. Angiosarcoma is an aggressive rapidly growing tumor that is typically fatal. Tumor successfully cleared after 14 weeks and normalization of skin cosmesis, texture, and pigmentation was achieved within 24 weeks.

### DETAILED DESCRIPTION OF THE INVENTION

The present disclosure may be understood more readily by reference to the following detailed description of desired embodiments and the examples included therein. In the following specification and the claims that follow, reference will be made to a number of terms which have the following meanings.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

All ranges disclosed herein are inclusive of the recited endpoint and independently combinable (for example, the range of “from 2 to 10” is inclusive of the endpoints, 2 and 10, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The present invention relates to compositions and methods for treating a neoplasia. Specifically, the invention relates to an off-label combinatorial composition for treating a neoplasia. The individual components of the composition are used in a manner and for diseases that are not otherwise on the product label of the components. The term “neoplasia,” as used herein refers to abnormal or uncontrolled cell growth. A “neoplasm”, or tumor or cancer, is an abnormal, unregulated, and disorganized proliferation of cell growth, and is generally referred to as cancer. A neoplasm may be benign or malignant. A neoplasm is malignant, or cancerous, if it has properties of destructive growth, invasiveness, and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

The inventors of the instant application surprisingly and unexpectedly found that a neoplasia such as a nonmelanoma skin cancer (e.g., basal cell carcinoma (BCC) and squamous cell carcinoma (SCC) and squamous cell carcinoma in situ (SCCIS)) can be effectively treated by the use of a combination of already FDA approved drugs, originally intended for other conditions and at much higher dosages. Specifically, the inventors of the instant application surprisingly and unexpectedly found that nonmelanoma skin cancer can be effectively treated by the use of a combination of imiquimod, diclofenac, hydrocortisone valerate, tretinoin, calcipotriene, celecoxib, and sirolimus, each at subtherapeutic doses. Moreover, Celecoxib and sirolimus are not conventionally administered topically and their use in this invention is novel.

Imiquimod was first approved by FDA in 1997 for treating genital warts. Imiquimod is a toll-like receptor 7 (TLR7) agonist and acts as an immune response modifier. TLR7 agonists including imiquimod are well known in the art. Any TLR7 agonist, known to one of skilled in the art, can be used in the invention described herein. Methods for making TLR7 agonists, including imiquimod, are well known in the art.

Diclofenac was approved by FDA in 1998 and has analgesic, anti-inflammatory, and antipyretic properties. Diclofenac is a non-steroidal anti-inflammatory drug (NSAID). Any NSAID, known to one of skilled in the art, can be used in the invention described herein. NSAIDs, including diclofenac, are well known in the art. Methods for making NSAIDs, including diclofenac, are also well known in the art.

Hydrocortisone valerate was approved by FDA in 1984. This drug is indicated for the relief of the inflammatory and pruritic manifestations in skin. Hydrocortisone valerate is a glucocorticoid anti-inflammatory agent. Any glucocorticoid anti-inflammatory agent, known to one of skilled in the art, can be used in the invention described herein. Glucocorticoid anti-inflammatory agents, including hydrocortisone valerate, are well known in the art. Methods for making Glucocorticoid anti-inflammatory agents, including hydrocortisone valerate, are also well known in the art.

Tretinoin was approved for medical use in 1962. This drug topically is used for the treatment of acne. Tretinoin is a vitamin A derivative. Any vitamin A derivative, known to one of skilled in the art, can be used in the invention described herein. Vitamin A derivatives, including tretinoin, are well known in the art. Methods for making vitamin A derivatives, including tretinoin, are also well known in the art.

Calcipotriene was approved for medical use in 1993. This medication is used to treat psoriasis. Calcipotriene is a form of vitamin D and a vitamin D3 derivative. It works by slowing down the growth of skin cells. Any vitamin D3 derivative, known to one of skilled in the art, can be used in the invention described herein. Vitamin D3 derivatives, including calcipotriene, are well known in the art. Methods for making vitamin D3 derivatives, including calcipotriene, are also well known in the art.

Celecoxib was approved for medical use in 1998. It is used orally to treat arthritis, acute pain, menstrual pain and discomfort, and familial polyposis. Celecoxib is also a non-steroidal anti-inflammatory drug (NSAID). As discussed above, any NSAID, known to one of skilled in the art, can be used in the invention described herein. NSAIDs, including celecoxib, are well known in the art. Methods for making NSAIDs, including celecoxib, are also well known in the art.

Sirolimus was approved for medical use in 1999. It is used to prevent organ transplant rejection, to coat coronary stents, and to treat a rare lung disease called lymphangioleiomyomatosis. Sirolimus is a mammalian target of rapamycin (mTOR) inhibitor. Any mTOR inhibitor, known to one of skilled in the art, can be used in the invention described herein. mTOR inhibitors, including sirolimus, are well known in the art. Methods for making mTOR inhibitors, including sirolimus, are also well known in the art.

In one embodiment, provided herein is a pharmaceutical composition to treat a neoplasia in a subject, the composition comprising: a therapeutically effective amount of a toll-like receptor 7 (TLR7) agonist (e.g., imiquimod), a non-steroidal anti-inflammatory drug (NSAID) (e.g., diclofenac, celecoxib), a glucocorticoid anti-inflammatory agent (e.g., hydrocortisone valerate), a vitamin A derivative (e.g., tretinoin), a vitamin D3 derivative (e.g., calcipotriene), a mechanistic target of rapamycin (mTOR) inhibitor (e.g., sirolimus), or a combination thereof, wherein each of said molecule is present in an amount effective to treat a neoplasia.

In some embodiments, a first pharmaceutical composition comprises a TLR7 agonist (e.g., imiquimod), a second pharmaceutical composition comprises an NSAID (e.g., diclofenac, celecoxib, or a combination thereof), a third pharmaceutical composition comprises a glucocorticoid anti-inflammatory agent (e.g., hydrocortisone valerate), a fourth pharmaceutical composition comprises a vitamin A derivative (e.g., tretinoin), a fifth pharmaceutical composition comprises a vitamin D3 derivative (e.g., calcipotriene), and a sixth pharmaceutical composition comprises a mTOR inhibitor (e.g., sirolimus). These individual and separate compositions may be administered independently or together.

The invention also provides pharmaceutical and biological compositions comprising the one or more therapeutic agents or molecules, discussed above, and one or more pharmaceutically acceptable carriers. “Pharmaceutically acceptable carriers” include any excipient which is nontoxic to the cell or mammal being exposed thereto at the dosages and concentrations employed. The pharmaceutical composition may include one or additional therapeutic agents.

Pharmaceutically acceptable carriers include solvents, dispersion media, buffers, coatings, antibacterial and antifungal agents, wetting agents, preservatives, buggers, chelating agents, antioxidants, isotonic agents and absorption delaying agents.

Pharmaceutically acceptable carriers include water; saline; phosphate buffered saline; dextrose; glycerol; alcohols such as ethanol and isopropanol; phosphate, citrate and other organic acids; ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; EDTA; salt forming counterions such as sodium; and/or nonionic surfactants such as TWEEN, polyethylene glycol (PEG), and PLURONICS; isotonic agents such as sugars, polyalcohols such as mannitol and sorbitol, and sodium chloride; as well as combinations thereof. Antibacterial and antifungal agents include parabens, chlorobutanol, phenol, ascorbic acid, and thimerosal.

The pharmaceutical compositions of the invention may be formulated in a variety of ways, including for example, liquid, semi-solid, solid dispersion, and solid dosage forms, or a combination thereof. Examples of a formulation include, for example, but not limited to, a liquid solution (e.g., topical solution, injectable solution), a dispersion or a suspension, a gel, a lotion, a cream, an ointment, a foam, a paste, a powder, a semisolid structure, an aerosol, a transdermal delivery vehicle, a tablet, a pill, a liposome and a suppository.

In some embodiments, the composition is in a form suitable for topical, transmucosal, transdermal, oral, intravenous, intraarterial, intramuscular, subcutaneous, or parenteral, administration. The composition may be formulated as an immediate, controlled, extended or delayed release composition.

In a particular embodiment, the pharmaceutical composition of the invention is a topical formulation. Suitable topical formulation forms include, for example, but not limited to, a gel, a lotion, a cream, an ointment, a foam, a paste, an aerosol, a transdermal delivery vehicle, and the like, as described, for example, in Remington: The Science and Practice of Pharmacy (21.sup.st Edition, University of the Sciences in Philadelphia, 2005). Ointments are semi-solid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. Creams are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant. Gels are semisolid, suspension-type systems. Single-phase gels contain organic macromolecules (polymers) distributed substantially uniformly throughout the carrier liquid, which is typically aqueous, but also, preferably, contain an alcohol such as ethanol or isopropanol and, optionally, an oil. In order to prepare a uniform gel, dispersing agents such as alcohol or glycerin can be added, or the gelling agent can be dispersed by trituration, mechanical mixing or stirring, or combinations thereof. Lotions are preparations to be applied to the skin surface without friction, and are typically liquid or semiliquid preparations in which solid particles, including the active agent, are present in a water or alcohol base. Lotions are usually suspensions of finely divided solids and will typically contain suspending agents to produce better dispersions as well as compounds useful for localizing and holding the active agent in contact with the skin. Pastes are semisolid dosage forms in which the active agent is suspended in a suitable base. Depending on the nature of the base, pastes are divided between fatty pastes or those made from single-phase aqueous gels.

Various additives, known to those skilled in the art, may be included in the topical formulations. For example, relatively small amounts of hydroxypropyl-beta-cyclodextrin, may be used to solubilize certain drug substances to create a solid dispersion. Other optional additives include opacifiers, antioxidants, fragrance, colorant, gelling agents, thickening agents, stabilizers, surfactants and the like. Other agents may also be added, such as antimicrobial agents, to prevent spoilage upon storage, i.e., to inhibit growth of microbes such as yeasts and molds. For those drugs having an unusually low rate of permeation through the skin or mucosal tissue, it may be desirable to include a permeation enhancer in the formulation. The formulation may also contain irritation-mitigating additives to minimize or eliminate the possibility of skin irritation or skin damage resulting from the drug, the enhancer, or other components of the dosage form. The formulations may also contain ether physiologically acceptable excipients or other minor additives, such as fragrances, dyes, emulsifiers, buffers, cooling agents (e.g. menthol), antibiotics, stabilizers or the like. In some instances, one component may serve more than one function.

Generally, dispersions are prepared by incorporating the active compound together or into a vehicle, which contains a basic dispersion medium and the required other ingredients from those enumerated above.

In one embodiment for mucosal application, the formulation may contain additives to enhance mucosoadhesion, spreadability, and rheological properties. Such additives may include poly-2-hydroxyethylmethacrylate, that when hydrated with the complex, can form a flexible film and act as a delivery mechanism to the skin. Other additives may include carboxymethylcellulose 10-35%, pectin 1-5%, and gelatin 2-10%.

In another embodiment, the composition includes a skin penetration enhancer that facilitates transcutaneous penetration of ingredients in the composition. Any skin penetration enhancer known to one of skilled in the art can be used. In an exemplary embodiment, the skin penetration enhancer is hyaluronate sodium. In another exemplary embodiment, the skin penetration enhancer is hydroxypropyl-beta-cyclodextrin, used, for example, in equimolar concentration with celecoxib.

In some embodiments, the composition includes isotonic agents, for example, sugars, polyalcohols, such as mannitol, sorbitol, or sodium chloride. Prolonged absorption of the compositions can be brought about by including in the composition an agent which delays absorption, for example, aluminum monostearate and gelatin.

Sterile solutions can be prepared by incorporating the molecules of the invention, in the required amount in an appropriate solvent with one or a combination of ingredients enumerated herein, as required, followed by filtered sterilization.

The preparations are processed and filled into containers and may also be sealed, according to methods known in the art. Further, the preparations may be packaged and sold in the form of a kit.

In one embodiment, the kit contains the individual components and an applicator that combines and mixes and delivers the needed amount as needed for each application, in real time.

Effective doses of the compositions of the present invention, for treatment of conditions or diseases as described herein vary depending upon many different factors, including means of administration, target site, physiological state of the patient, whether the patient is human or an animal, other medications administered, and whether treatment is prophylactic or therapeutic. Usually, the patient is a human but non-human subjects including transgenic mammals, and companion animals, can also be treated. Treatment dosages may be titrated using routine methods known to those of skill in the art to optimize safety and efficacy.

In some embodiments, the dosage is determined based on a personalized medicine algorithm known to one skilled in the art, by which therapeutic efficacy can be achieved without undesirable irritation and inflammation using the combination of agents. Specifically, this is in marked contrast to the current practice and belief that topical treatment of skin cancer requires intense local inflammation for effective treatment. This is also in contrast to the current practice and belief specifically that a local inflammatory reaction is requisite in the mechanism of action of chemotherapy or imiquimod. For example, when used as monotherapy according to the package label recommended usage, the incidence of inflammation in the imiquimod clinical studies was as high as 97%.

In some embodiments, the dosage based on an algorithm, known in the art, varies from twice a week to twice a day application. The dose frequency is started at the least frequent level and increased at regular intervals, for example every 1-2 weeks, if there is no discomfort and no evidence of gross undesirable inflammation.

In some embodiments, the dosage according to a protocol, known in the art, can be guided by a healthcare provider skilled in the art who is in direct observation with the user. In another embodiment, the dosage can be guided by a doctor skilled in the art using photographic images of the treated site along with responses to defined questions querying symptoms, taken by the user or by a healthcare provider and sent electronically to an experienced healthcare provider skilled in the art, for example at a centralized service location. In yet another embodiment, the dosage can be guided by photographic images of the treated site along with responses to defined questions querying symptoms that are analyzed and classified automatically using computer image analysis algorithms, such as artificial intelligence, to generate a recommendation to maintain or increase the frequency of the dose, according to the protocol and a novel taxonomy of tissue responses. The capability of using computers to automatically and accurately analyze and partition disease using image pixels and deep learning algorithms has recently been shown (Nature 2017; 542:115).

In one example, the composition comprises a therapeutically effective amount of a toll-like receptor 7 (TLR7) agonist (e.g., imiquimod), a non-steroidal anti-inflammatory drug (NSAID) (e.g., diclofenac, celecoxib, or both), a glucocorticoid anti-inflammatory agent (e.g., hydrocortisone valerate), a vitamin A derivative (e.g., tretinoin), a vitamin D3 derivative (e.g., calcipotriene), a mechanistic target of rapamycin (mTOR) inhibitor (e.g., sirolimus), or a combination thereof, wherein each of said molecule is present in an amount effective to treat a neoplasia.

In one embodiment, the composition comprises imiquimod, calcipotriene, tretinoin, diclofenac, hydrocortisone valerate, celecoxib, sirolimus, or a combination thereof. The combination of agents exerts actions unique from their actions on FDA approved disease indications, including antiangiogenic and immunotherapeutic activity. For example, in one aspect, the combination of agents confers antiangiogenic activity. In one example, imiquimod upregulates endogenous interferon-alpha, interferon-beta, and interferon-gamma that downregulates endothelial integrins, inhibits endothelial cell proliferation, migration, and invasion, and increases endothelial cell apoptosis. In another example, imiquimod upregulates interleukin-12 that decreases production of bFGF and IL-8 and increases interferon-gamma via T cells and NK cells. In yet another example, imiquimod upregulates interleukin-18 that suppresses angiogenesis.

In another aspect, the combinations of agents confer immunotherapeutic activity. For example, celecoxib inhibits COX-2 which is implicated in conferring resistance to immune detection by cancers. In another example, sirolimus fosters cancer immunotherapy by preserving T regulatory cells selectively. In yet another example, imiquimod activates the innate immune system through peritumoral and intratumoral infiltration by macrophages and neutrophils, which subsequently resulting in T cell activation.

A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. Importantly, a therapeutically effective concentration of a drug may be lowered when it is used in combination with another drug or drugs. For example, a drug used at a subtherapeutic concentration may unexpectedly have therapeutic effects when used in combination. A therapeutically effective amount is also one in which any toxic or detrimental effects of the molecule are outweighed by the therapeutically beneficial effects. This invention creates therapeutic outcomes at surprisingly low subtherapeutic doses. In one embodiment, the composition comprises a therapeutically effective amount of imiquimod, calcipotriene, tretinoin, diclofenac, hydrocortisone valerate, celecoxib, and sirolimus that is substantially lower than the concentrations used in the commercially available approved agents. In an example, said imiquimod is present at the concentration ranging from about 0.1% (w/w) to about 5% (w/w); said calcipotriene is present at the concentration ranging from about 0.0001% (w/w) to about 0.005% (w/w); said tretinoin is present at the concentration ranging from about 0.001% (w/w) to about 0.1% (w/w); said diclofenac is present at the concentration ranging from about 0.05% (w/w) to about 3% (w/w); said hydrocortisone valerate is present at the concentration ranging from about 0.005% (w/w) to about 0.25% (w/w); said celecoxib is present at the concentration ranging from about 0.1% (w/w) to about 10% (w/w); or said sirolimus is present at the concentration ranging from about 0.01% (w/w) to about 1% (w/w).

In one embodiment, said imiquimod is present at the concentration of about 1% (w/w); said calcipotriene is present at the concentration of about 0.001% (w/w); said tretinoin is present at the concentration of about 0.02% (w/w); said diclofenac is present at the concentration of about 0.6% (w/w); or said hydrocortisone valerate is present at the concentration of about 0.04% (w/w).

In another embodiment, said imiquimod is present at the concentration of about 0.833% (w/w); said calcipotriene is present at the concentration of about 0.00083% (w/w); said tretinoin is present at the concentration of about 0.0167% (w/w); said diclofenac is present at the concentration of about 0.5% (w/w); or said hydrocortisone valerate is present at the concentration of about 0.0033% (w/w).

In another embodiment, said imiquimod is present at the concentration of about 0.71% (w/w); said calcipotriene is present at the concentration of about 0.0007% (w/w); said tretinoin is present at the concentration of about 0.014% (w/w); said diclofenac is present at the concentration of about 0.43% (w/w); said hydrocortisone valerate is present at the concentration of about 0.029% (w/w); said celecoxib is present at the concentration of about 2% (w/w); or said sirolimus is present at the concentration of about 0.014% (w/w).

In another embodiment, said imiquimod is present at the concentration of about 0.313% (w/w); said calcipotriene is present at the concentration of about 0.000313% (w/w); said tretinoin is present at the concentration of about 0.00625% (w/w); said diclofenac is present at the concentration of about 0.1875% (w/w); said hydrocortisone valerate is present at the concentration of about 0.0125% (w/w); said celecoxib is present at the concentration of about 2% (w/w); or said sirolimus is present at the concentration of about 0.0625% (w/w).

In yet another embodiment, said imiquimod is present at the concentration of about 0.833% (w/w); said calcipotriene is present at the concentration of about 0.00083% (w/w); said tretinoin is present at the concentration of about 0.0167% (w/w); said diclofenac is present at the concentration of about 0.5% (w/w); said hydrocortisone valerate is present at the concentration of about 0.0033% (w/w); or said celecoxib is present at the concentration of about 2% (w/w).

The invention further provides a kit comprising a therapeutically effective amount of a toll-like receptor 7 (TLR7) agonist (e.g., imiquimod), a non-steroidal anti-inflammatory drug (NSAID) (e.g., diclofenac, celecoxib, or both), a glucocorticoid anti-inflammatory agent (e.g., hydrocortisone valerate), a vitamin A derivative (e.g., tretinoin), a vitamin D3 derivative (e.g., calcipotriene), a mechanistic target of rapamycin (mTOR) inhibitor (e.g., sirolimus), or a combination thereof.

The invention further provides methods of treating a disease or condition, comprising administering to a subject in need thereof a therapeutically effective amount of a toll-like receptor 7 (TLR7) agonist (e.g., imiquimod), a non-steroidal anti-inflammatory drug (NSAID) (e.g., diclofenac, celecoxib, or both), a glucocorticoid anti-inflammatory agent (e.g., hydrocortisone valerate), a vitamin A derivative (e.g., tretinoin), a vitamin D3 derivative (e.g., calcipotriene), a mechanistic target of rapamycin (mTOR) inhibitor (e.g., sirolimus), or a combination thereof.

As used herein, the terms “treat” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition prior to other treatments such as surgery, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable, and prevention of disease recurrence. “Treatment” can also mean prolonging survival as compared to expected survival if not receiving treatment. Those in need of treatment include those already with the disease or condition as well as those prone to having the disease or condition or those in which the disease or condition is to be prevented.

A disease or condition treated by the invention includes, for example, neoplasia. The neoplasia may be present in adrenal gland, anus, auditory nerve, bile duct, bladder, bone, brain, breast, central nervous system, cervix, colon, ear, endometrium, esophagus, eye, eyelids, fallopian tube, gastrointestinal tract, head and neck, heart, kidney, larynx, liver, lung, mandible, mandibular condyle, maxilla, mouth, nasopharynx, nose, oral cavity, ovary, pancreas, parotid gland, penis, pinna, pituitary, prostate gland, rectum, retina, salivary gland, skin, small intestine, spinal cord, stomach, testes, thyroid, tonsil, urethra, uterus, vagina, vestibulocochlear nerve and vulva neoplasms, lymph, or lymph node.

In one embodiment, the neoplasia is a solid tumor. In another embodiment, the neoplasia is not a solid tumor.

In an exemplary embodiment, the neoplasia is associated with a lesion. In one example, the lesion is a pre-malignant lesion. In another example, lesion is a normal tissue at a risk of transforming into malignancy. In another example, the lesion is in a tissue in the setting of immuno suppression.

The lesion can be hidden or undetected lesion. The composition of the invention is capable of facilitating the detection of said hidden or undetected lesion. In some embodiments, the lesion is malignant, for example, malignant skin cancer.

Cancers/tumors which may be treated by the invention include any cancer or tumor. Examples of cancers/tumors which may be treated include, but are not limited to, melanoma and non-melanoma skin cancer (NMSC). Examples of melanoma include, for example, but not limited to, lentigo maligna melanoma, superficial spreading melanoma, acral lentiginous melanoma, mucosal melanoma, nodular melanoma, polypoid melanoma, desmoplastic melanoma, small-cell melanoma, spitzoid melanoma, uveal melanoma (including choroidal melanoma, ciliary body melanoma, or iris melanoma), amelanotic melanoma, and nevoid melanoma. Examples of melanoma related tumors include, for example, but not limited to, conventional atypical Spitz tumor, superficial atypical Spitz tumor, borderline deep penetrating nevus-like lesion, and nevoid borderline tumor.

Examples of NMSC include, for example, but not limited to, basal cell carcinoma (BCC), squamous cell carcinoma in situ (SCCIS), squamous cell carcinoma (SCC), an angiosarcoma, cutaneous B-cell lymphoma, cutaneous T-cell lymphoma, dermatofibrosarcoma, dermatofibrosarcoma protuberans, Merkel cell carcinoma, Kaposi's sarcoma, or sebaceous carcinoma.

Methods of treating cancer include, but are not limited to, e.g., inhibiting angiogenesis in the tumor, inhibiting tumor growth, inhibiting tumor cell migration, proliferation, or invasion, promoting tumor cell apoptosis, and promoting immune clearance of tumor cells.

Cancers to be treated include primary tumors and secondary or metastatic tumors (including those metastasized from lung, breast, or prostate), as well as recurrent or refractory tumors. Recurrent tumors encompass tumors that appear to be inhibited by treatment, but recur after a period of time. Refractory tumors are tumors that have failed to respond or are resistant to treatment with one or more conventional therapies for the particular tumor type. Refractory tumors include those that are refractory to treatment with one or more destructive modalities, including surgery, radiation, cryotherapy, electrodessication and curettage; or chemotherapeutic agents, or hormone therapy, immune response modifying agents, or signal targeting pathway agents.

Therapy may be “first-line”, i.e., as an initial treatment in patients who have had no prior anti-cancer treatments, either alone or in combination with other treatments; or “second-line”, as a treatment in patients who have had one prior anti-cancer treatment regimen, either alone or in combination with other treatments where initial treatment with conventional therapies have failed and there is residual tumor or recurrent tumor; or as “third-line”, “fourth-line”, etc. treatments, either alone or in combination with other treatments. Therapy may also be neo-adjuvant prior to surgery, to allow for smaller surgical margins and smaller surgical scars.

Therapy may also be given to patients who have had previous treatments which have been partially successful but are intolerant to the particular treatment. Therapy may also be given as an adjuvant treatment, i.e., to prevent reoccurrence of cancer in patients with no currently detectable disease or after surgical removal of tumor. Therapy may also be given to patients who are not candidates for conventional therapy due to age or comorbidities for example. Therapy may also be given to patients who refuse conventional modalities.

Cancers that may be treated include tumors that are not vascularized, or not yet substantially vascularized, as well as vascularized tumors. The cancers may be comprised of non-solid tumors (such as leukemias and lymphomas) or may be solid tumors. Types of cancers to be treated with the antibodies of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are included.

More than one therapeutic agent of the invention may be administered, either incorporated into the same composition or administered as separate compositions.

A therapeutic agent of the invention may be administered alone, or in combination with one or more therapeutically effective agents or treatments. The other therapeutically effective agent may be conjugated to the therapeutic agent of the invention, incorporated into the same composition as the therapeutic agent, or may be administered as a separate composition. The other therapeutically agent or treatment may be administered prior to, during and/or after the administration of the therapeutic agent.

In one embodiment, the therapeutic agents of the invention are co-administered. In another embodiment, one therapeutic agent of the invention is administered independently from another therapeutic agent of the invention. In one embodiment, one therapeutic agent of the invention is administered first, followed by the administration of another therapeutic agent of the invention.

Other therapeutically effective agents/treatments include surgery, anti-neoplastics (including chemotherapeutic agents and radiation), anti-angiogenesis agents, antibodies to other targets, small molecules, photodynamic therapy, immunotherapy, cytotoxic agents, cytokines, chemokines, growth inhibitory agents, anti-hormonal agents, kinase inhibitors, cardioprotectants, immunotherapeutic agents, agents that promote proliferation of hematological cells, and protein tyrosine kinase (PTK) inhibitors, and other signal transduction inhibitors.

A chemotherapeutic agent may be administered as a prodrug. The term “prodrug” refers to a precursor or derivative form of a pharmaceutically active substance that is less cytotoxic to tumor cells compared to the parent drug and is capable of being enzymatically activated or converted into the more active parent form. The prodrugs that may find use with the compositions and methods as provided herein include but are not limited to phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug.

The administration of the therapeutic agents or the composition of the invention and/or treatments may occur simultaneously, or separately, via the same or different route, at the same or different times. Dosage regimens may be adjusted to provide the optimum desired response (e.g., a therapeutic or prophylactic response).

In one example, a single bolus may be administered. In another example, several divided doses may be administered over time. In yet another example, a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of active compound calculated to produce a desired therapeutic effect. In some embodiments, the dosage unit forms of the invention are dictated by and directly dependent on the unique characteristics of the active compound and the particular therapeutic or prophylactic effect to be achieved.

The composition of the invention may be administered only once, or it may be administered multiple times. For multiple dosages, the composition may, for example, be administered twice a day, once a day, five days a week, once every two days, three times a week, twice a week, weekly, once every two weeks, or monthly, or any combination of such dose frequencies. The composition of the invention may also be covered under a dressing after application to enhance absorption and tissue response.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

“Administration” to a subject is not limited to any particular delivery system and may include, without limitation, topical, transdermal, parenteral (including subcutaneous, intravenous, intramedullary, intraarticular, intramuscular, or intraperitoneal injection) rectal, or oral (for example, in capsules, suspensions or tablets). Administration to a host may occur in a single dose or in repeat administrations, and in any of a variety of physiologically acceptable salt forms, and/or with an acceptable pharmaceutical carrier and/or additive as part of a pharmaceutical composition (described earlier). Once again, physiologically acceptable salt forms and standard pharmaceutical formulation techniques are well known to persons skilled in the art (see, for example, Remington's Pharmaceutical Sciences, Mack Publishing Co.).

As used herein, a “composition” refers to any composition that contains a pharmaceutically effective amount of one or more therapeutic agents of the invention (e.g., imiquimod, diclofenac, hydrocortisone valerate, tretinoin, calcipotriene, celecoxib, sirolimus).

The methods of treatment described herein can be used to treat any suitable subject, including primates, such as monkeys and humans, horses, cows, cats, dogs, birds, aquatic animals, rabbits, and rodents such as rats and mice. In one embodiment, the subject to be treated is a mammal, for example, a human.

All patents and literature references cited in the present specification are hereby incorporated by reference in their entirety.

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### EXAMPLES

### Example 1

### Composition I

The form of Composition I is a semisolid topical dispersion in which ointment, cream, and gel formulation are combined. Composition I includes a combination of imiquimod, diclofenac, hydrocortisone valerate, tretinoin, and calcipotriene.

One embodiment that has been tested in clinical studies, wherein said imiquimod is present at the concentration of about 1% (w/w); said calcipotriene is present at the concentration of about 0.001% (w/w); said tretinoin is present at the concentration of about 0.02% (w/w); said diclofenac is present at the concentration of about 0.6% (w/w); and said hydrocortisone valerate is present at the concentration of about 0.04% (w/w).

In Composition I and related compositions, the skin penetration enhancers are hyaluronate used at a concentration between about 0.4% to 0.5% and/or hydroxypropyl-beta-cyclodextrin (used with celecoxib) at a concentration between about 4% to 10%.

Advantages of the combination therapy is that each individual agent is used at a subtherapeutic level which minimizes any undesirable local side effects, but in combination the agents have additive and synergistic effects that have shown great efficacy and tolerability.

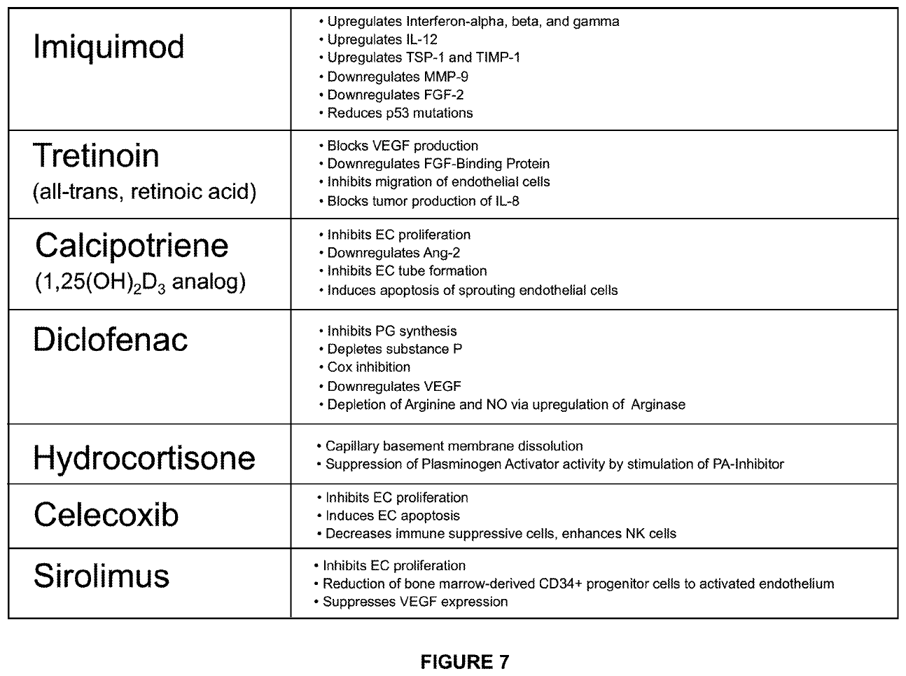

Each drug targets specific antiangiogenic and immunotherapeutic mechanisms (see FIG. 7), for example regulating interferons, IL-12, RAR-alpha, endothelial apoptosis, COX-2 mediated VEGF production, and basement membrane disruption. Examples of additive and synergistic effects on blood vessels include the following: interferon, induced by imiquimod and retinoids (tretinoin) make endothelial cells refractory to stimuli (for example the cytokine IL-8), and drive T-cell immune responses against cancer cells. Retinoids (tretinoin) and 1,25-D3 (calcipotriene) inhibit tenascin-C, a glycoprotein that regulates angiogenesis and mediate immune function. 1,25-D3 (calcipotriene) potentiates the effect of interleukin-12 (which is induced by imiquimod), which is antiangiogenic as well as T cell activating for anti-tumor effects. Sirolimus is antiangiogenic through suppression of angiogenesis pathways such as VEGF, and enhances cancer immunotherapy by modulating T regulatory cells and dendritic cells. Sirolimus in combination with COX-2 inhibition (celecoxib) results in enhanced antitumor effects by downregulating the mTOR pathway. Combining drug components with antiangiogenic and immunotherapeutic effects creates synergistic anti-tumor activity.

### Example 2

### Composition I-M (Mucosoadhesive)

Composition I-M refers to a semisolid topical dispersion of Composition I in a form that is used on mucosal surfaces such as in the mouth, or other internal orifice of the body.

One embodiment that has been used in clinical studies, said imiquimod is present at the concentration of about 0.8333% (w/w); said calcipotriene is present at the concentration of about 0.00083% (w/w); said tretinoin is present at the concentration of about 0.01667% (w/w); said diclofenac is present at the concentration of about 0.5% (w/w); and said hydrocortisone valerate is present at the concentration of about 0.0333% (w/w).

A paste form for mucosal use enhances adhesion, spreadability, and rheological properties of the topical complex. In one embodiment, the paste form which can be used safely in the mouth is created by adding poly-2-hydroxyethylmethacrylate, such as in the form of amlexanox 0.83333%.

In another embodiment, the paste form that is safe for use in the mouth is created by adding carboxymethylcellulose 10-30%, pectin 1-5%, and gelatin 2-10%.

### Example 3

### Composition II

The form of Composition II is a combination that includes a combination of imiquimod, diclofenac, hydrocortisone valerate, tretinoin, calcipotriene, and celecoxib. The form is a semisolid topical dispersion in which ointment, cream, and gel formulation are combined. In one embodiment of this and related compositions, said imiquimod is present at the concentration of about 0.89% (w/w); said calcipotriene is present at the concentration of about 0.00089% (w/w); said tretinoin is present at the concentration of about 0.018% (w/w); said diclofenac is present at the concentration of about 0.54% (w/w); said hydrocortisone valerate is present at the concentration of about 0.036% (w/w), and said celecoxib is present at the concentration of about 2.1% (w/w).

In Composition II and related compositions, the skin penetration enhancers are hyaluronate used at a concentration between about 0.4% to 0.5% and hydroxypropyl-beta-cyclodextrin (used with celecoxib) at a concentration between about 4% to 8%.

### Example 4

### Composition II-M (Mucosoadhesive)

Composition II-M refers to a semisolid topical dispersion of Composition II in a form that is used on mucosal surfaces such as in the mouth, or other internal orifice of the body.

One embodiment that has been used in clinical studies, said imiquimod is present at the concentration of about 0.62% (w/w); said calcipotriene is present at the concentration of about 0.00062% (w/w); said tretinoin is present at the concentration of about 0.012% (w/w); said diclofenac is present at the concentration of about 0.37% (w/w); and said hydrocortisone valerate is present at the concentration of about 0.025% (w/w), and said celecoxib is present at the concentration of about 2.1% (w/w).

A paste form for mucosal use enhances adhesion, spreadability, and rheological properties of the topical complex. In one embodiment, the paste form which can be used safely in the mouth is created by adding poly-2-hydroxyethylmethacrylate, such as in the form of amlexanox 0.55%. In this embodiment, said imiquimod is present at the concentration of about 0.55% (w/w); said calcipotriene is present at the concentration of about 0.00055% (w/w); said tretinoin is present at the concentration of about 0.011% (w/w); said diclofenac is present at the concentration of about 0.33% (w/w); and said hydrocortisone valerate is present at the concentration of about 0.022% (w/w), and said celecoxib is present at the concentration of about 2.1% (w/w).

In another embodiment, the paste form that is mucoadherent and is safe for use in the mouth is created by adding carboxymethylcellulose 10-20%, pectin 1-5%, and gelatin 2-10%.

### Example 5

### Composition III

The form of Composition III is a combination that includes a combination of imiquimod, diclofenac, hydrocortisone valerate, tretinoin, calcipotriene, celecoxib, and sirolimus. The form is a semisolid topical dispersion in which ointment, cream, and gel formulation are combined. In one embodiment of this and related compositions, said imiquimod is present at the concentration of about 0.72% (w/w); said calcipotriene is present at the concentration of about 0.00072% (w/w); said tretinoin is present at the concentration of about 0.014% (w/w); said diclofenac is present at the concentration of about 0.43% (w/w); said hydrocortisone valerate is present at the concentration of about 0.029% (w/w); said celecoxib is present at the concentration of about 2.1% (w/w); and said sirolimus is present at the concentration of about 0.014% (w/w).

In Composition III and related compositions, the skin penetration enhancers are hyaluronate used at a concentration of 0.3-0.5% and hydroxypropyl-beta-cyclodextrin (used with celecoxib) at a concentration between about 5% to 10%.

### Example 6

### Treating Basal Cell Carcinoma (BCC)

Composition I and Composition II and Composition III successfully treat basal cell carcinoma (BCC) of the skin. Composition I composition described in Example 1 was used to treat BCC in patients who were unable to undergo or refused conventional treatment modalities were treated topically on an individual basis. Frequency of administration was determined by a dosing algorithm. 136 lesions were treated topically, of which, 76 lesions were BCC lesions. Age of patients ranged from 30-90 years old. Treatment duration was 14 weeks. Both superficial and nodular BCCs were treated.

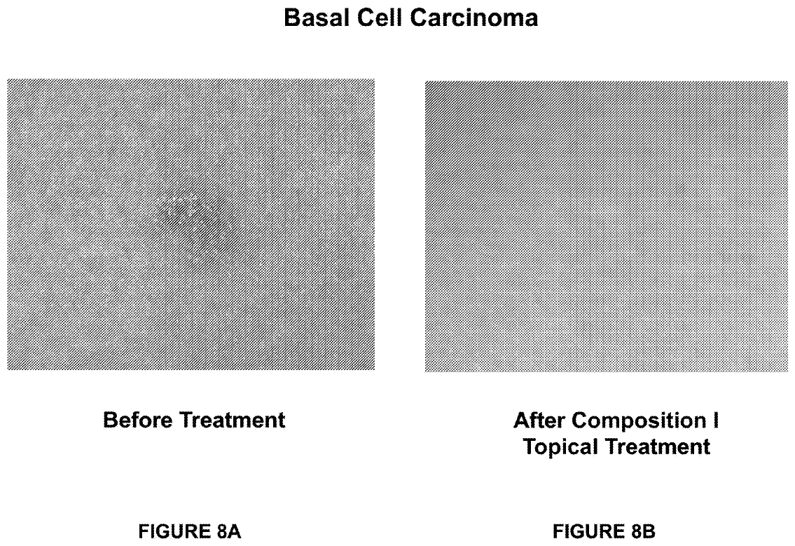

FIGS. 8A and 8B show the treatment of basal cell carcinoma. FIG. 8A shows the skin before treatment and FIG. 8B shows the skin after treatment.

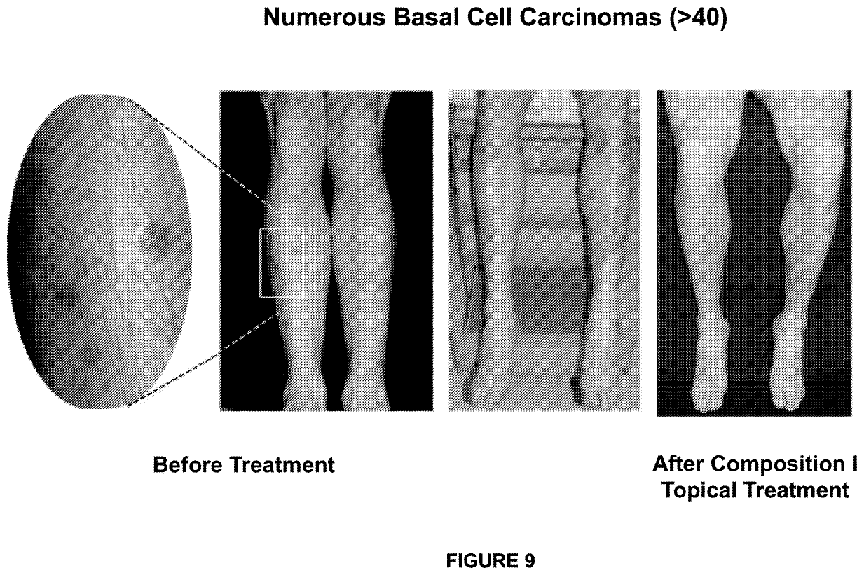

FIG. 9 shows treatment of numerous basal cell carcinomas (>40). Two pictures on the left show before treatment and the picture on the right shows after treatment.

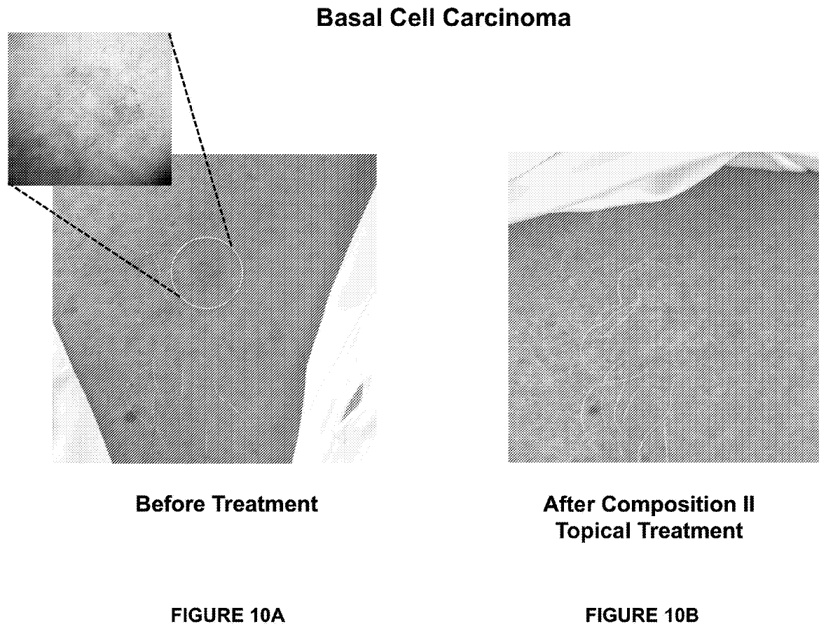

FIGS. 10A and 10B show the treatment of basal cell carcinoma on sun-damaged skin at risk for field cancerization.

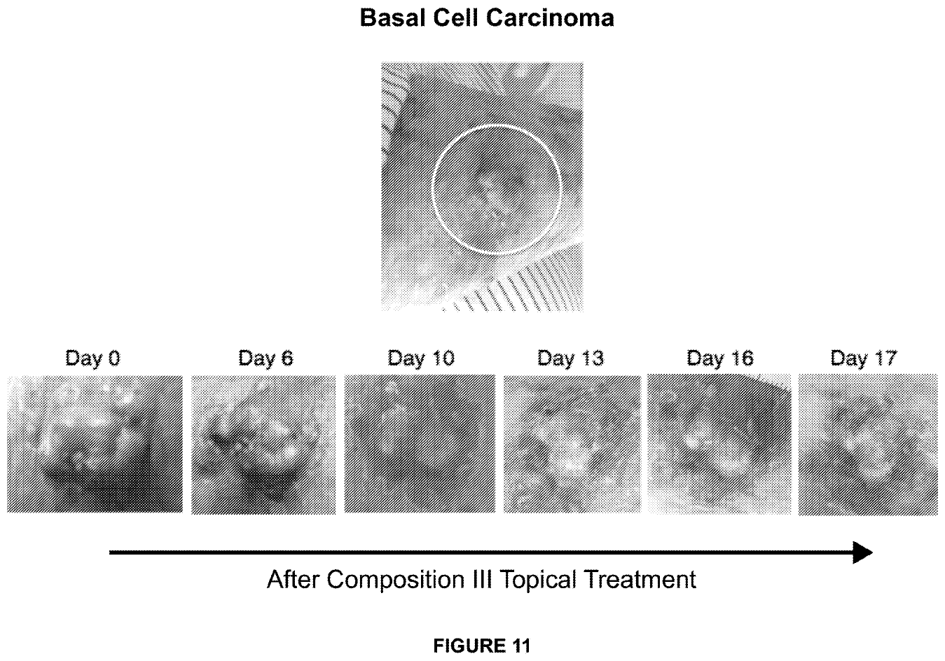

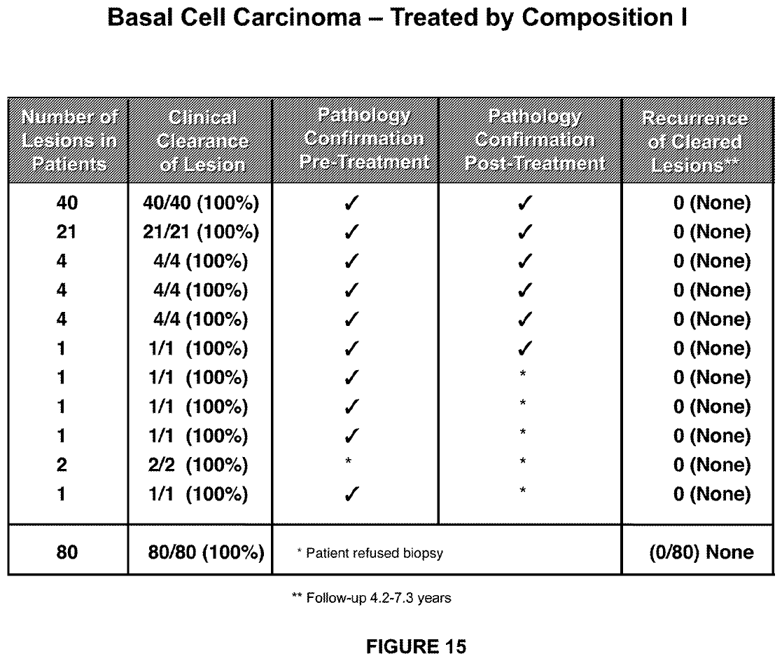

As shown in FIG. 11, treatment effects monitored on days 0, 6, 10, 13, 16, and 17 demonstrate rapid cancer regression. FIG. 15 shows a table of clinical results.

All BCCs successfully cleared with topical combinatorial treatment without any undesirable local reactions. The results fully demonstrate that the combinatorial composition is effective in treating BCC.

### Example 7

### Treating Squamous Cell Carcinoma In Situ (SCCIS)

Composition I and Composition II and Composition III successfully treat squamous cell carcinoma in situ (SCCIS) of the skin. Compositions described in Example 1, 3, and 5 were used to treat SCCIS of the skin in patients who were unable to undergo or refused conventional treatment modalities were treated topically on an individual basis. Frequency of administration was determined by a dosing algorithm. Of the 180 lesions treated topically, 36 lesions were SCCIS lesions. Age of patients ranged from 49-88 years old. Treatment duration ranged from 14-18 weeks.

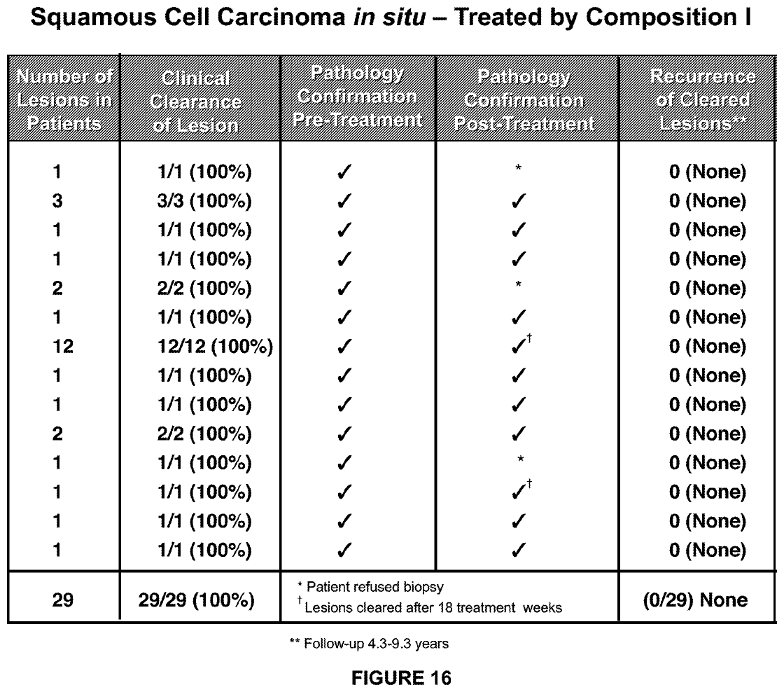

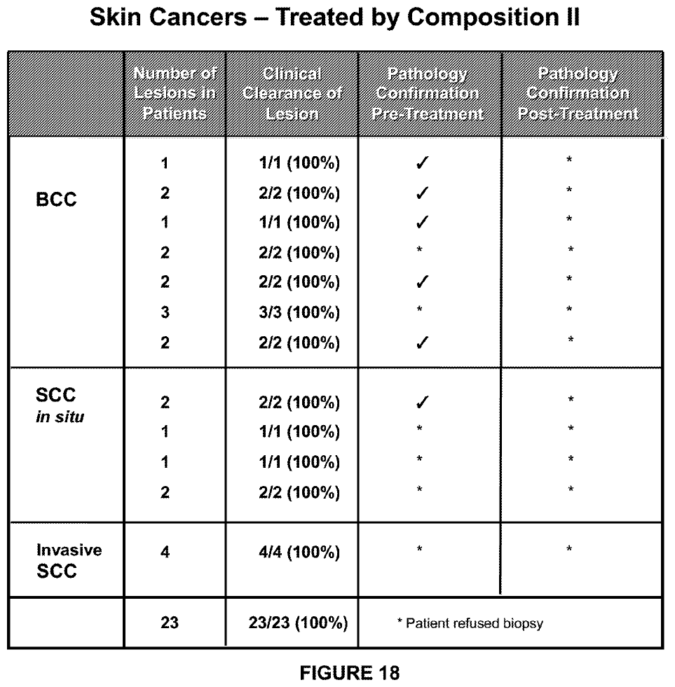

FIG. 16 and FIG. 18 show summary table on clinical results for treating SCC in situ.

All SCCISs successfully cleared with topical combinatorial treatment without any undesirable local reactions. The results fully demonstrate that the combinatorial composition is effective in treating SCC in situ.

### Example 8

### Treating Invasive Squamous Cell Carcinoma (SCC)

Composition I and Composition II and Composition III successfully treat invasive squamous cell carcinoma (SCC) of the skin. Compositions described in Example 1, 3, and 5 were used to treat SCC of the skin in patients who were unable to undergo or refused conventional treatment modalities were treated topically on an individual basis. Frequency of administration was determined by a dosing algorithm. Of the 180 lesions were treated topically, 46 lesions were invasive SCC lesions. Age of patients ranged from 41-88 years old. Treatment duration was 14 weeks.

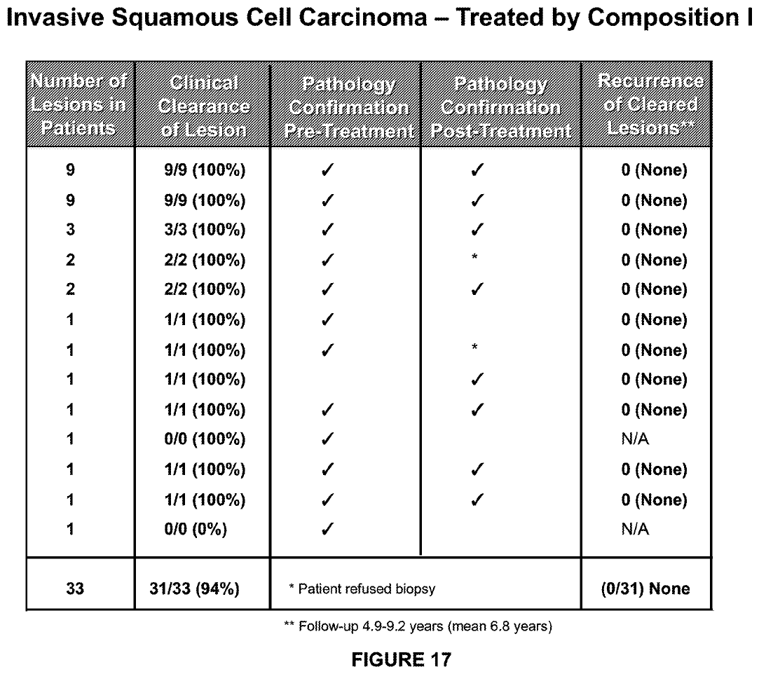

FIG. 17 and FIG. 18 and FIG. 19 show summary tables on clinical results for treating invasive SCC.

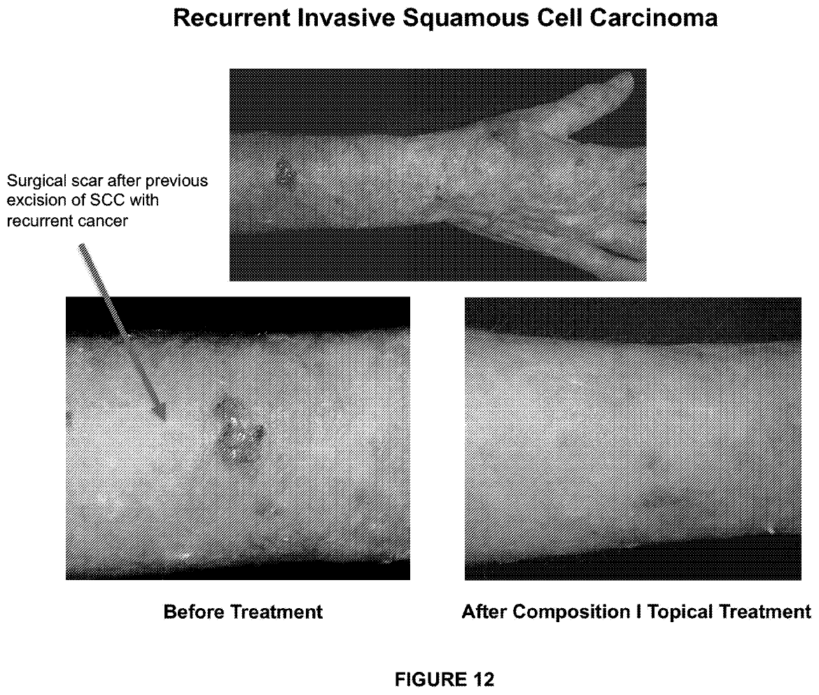

FIG. 12 shows the treatment of recurrent, invasive squamous cell carcinoma after failed surgical treatment. Bottom left picture shows recurrent cancer arising within surgical scar. Bottom right shows clearance after topical treatment.

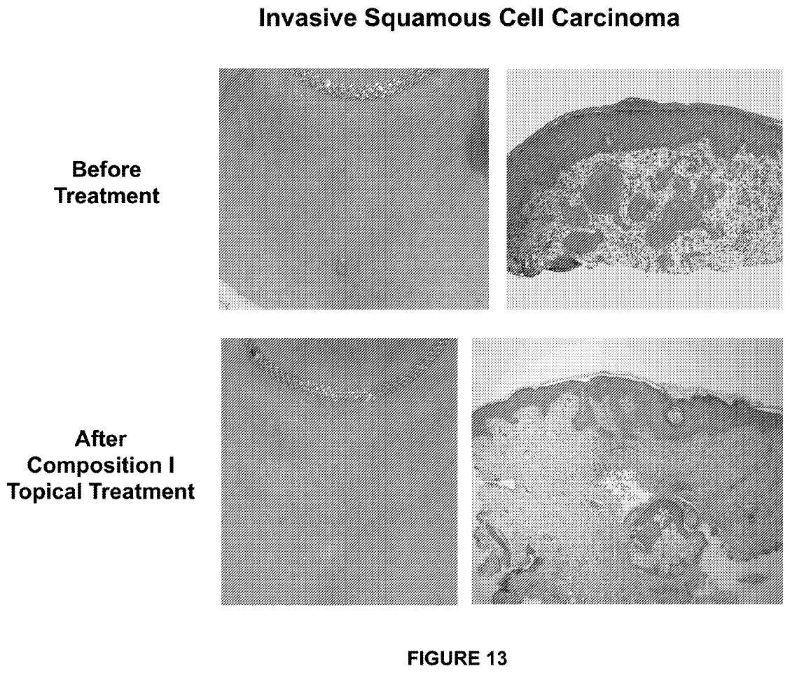

In addition, FIG. 13 shows the treatment of invasive squamous cell carcinoma. Top pictures show the clinical and histopathological images of the tumor before treatment. Bottom pictures shows after treatment with complete elimination of tumor.

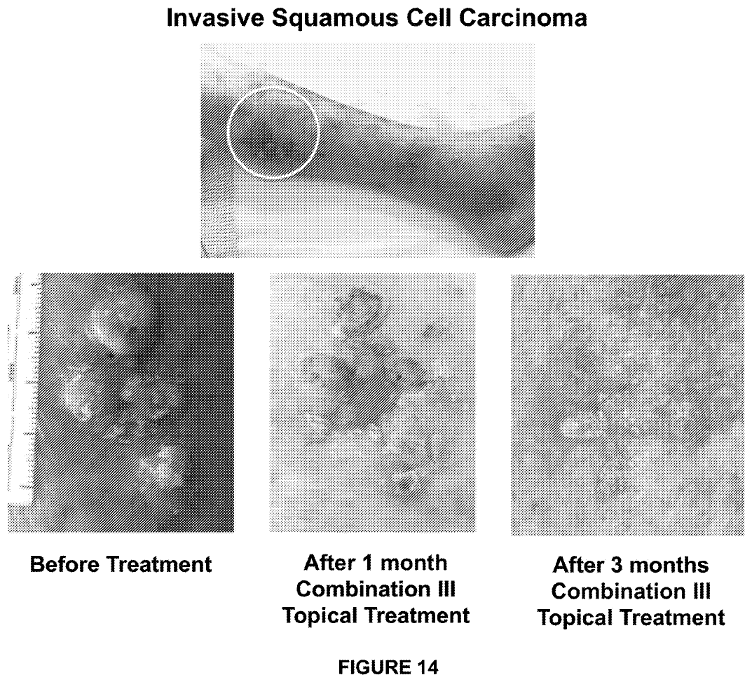

Furthermore, FIG. 14 also shows the treatment of invasive squamous cell carcinoma. Top picture and bottom left picture shows tumor before treatment. Bottom middle picture shows significant interval improvement after 1 month of treatment. Bottom right picture shows tumor clearance after 3 months of topical treatment.

Overall, invasive SCCs had 96% clearance with topical combinatorial treatment without any undesirable local reactions. Composition I showed 94% clearance, while Composition II and Composition III showed complete elimination of tumor.

The results fully demonstrate that the combinatorial composition is effective in treating invasive SCC.

### Example 9

### Efficacy in Treating Skin Cancers

The topical combination composition was used for treating basal cell carcinoma (BCC), squamous cell carcinoma in situ (SCCIS) and invasive SCC (SCC), based on a multi-targeting combinatorial approach utilizing FDA-approved drugs. The regimen included Composition II and III described in Example 3 and 5. Patients who were unable to undergo or refused conventional treatment modalities were treated on an individual basis using Composition II and III described in Example 6, 7, and 8. Frequency of administration was determined by a dosing algorithm.

38 lesions were treated topically with Composition II and Composition III, of which, 18 lesions were BCC, 7 lesions were SCCIS lesions and 13 lesions were invasive SCC lesions. Treatment duration was 14 weeks.

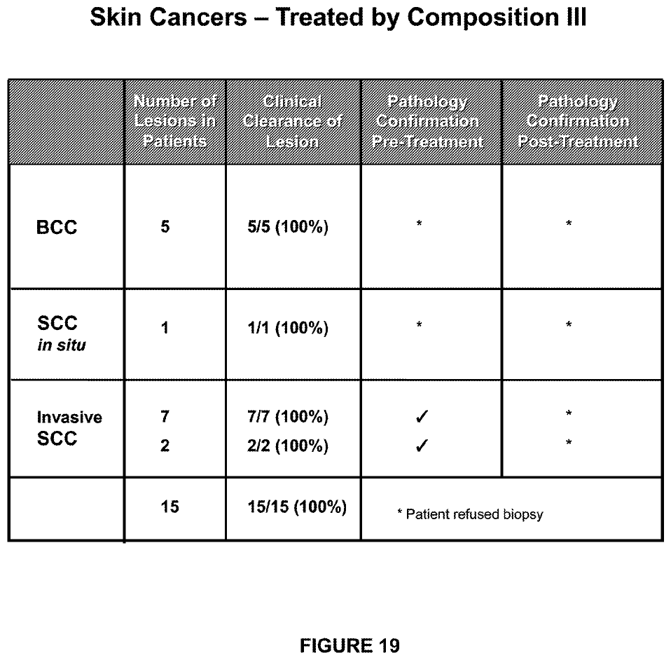

FIG. 18 and FIG. 19 show summary tables of clinical results for treating skin cancers (basal cell carcinoma (BCC); squamous cell carcinoma in situ (SCCIS); and invasive squamous cell carcinoma (SCC)) with the use of Composition II and Composition III. All skin cancers successfully cleared with topical combinatorial treatment without any undesirable local reactions.

The results fully demonstrate that Composition II and III and related off-label combinatorial compositions are effective in treating BCC, SCCIS, and invasive SCC.

### Example 10

### Effect on Tumor Microvessel Density

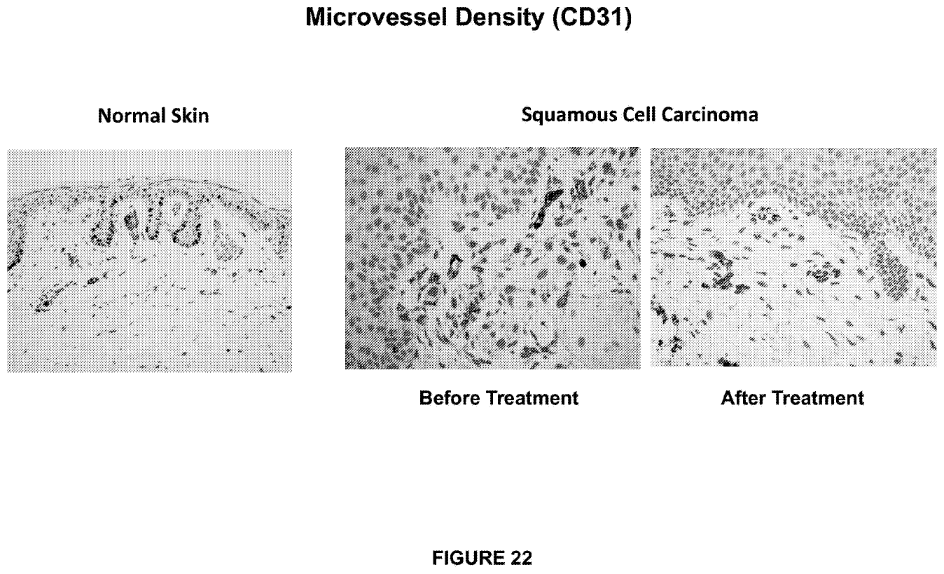

FIG. 22 shows the effect on tumor microvessel density (CD31) by Composition I, described in Example 1. Microvessel density (MVD) was measured by staining for CD31 and counting hotspots. There was increased MVD in SCC tumor stroma compared to normal skin controls. Antiangiogenic treatment with Composition I showed a statistical trend towards decreased MVD (Pre-treatment average MVD=36.0 versus Post-treatment average MVD=19.8). Figure on far left shows MVD of normal skin, middle figure shows MVD of SCC before treatment, and figure on far right shows after treatment.

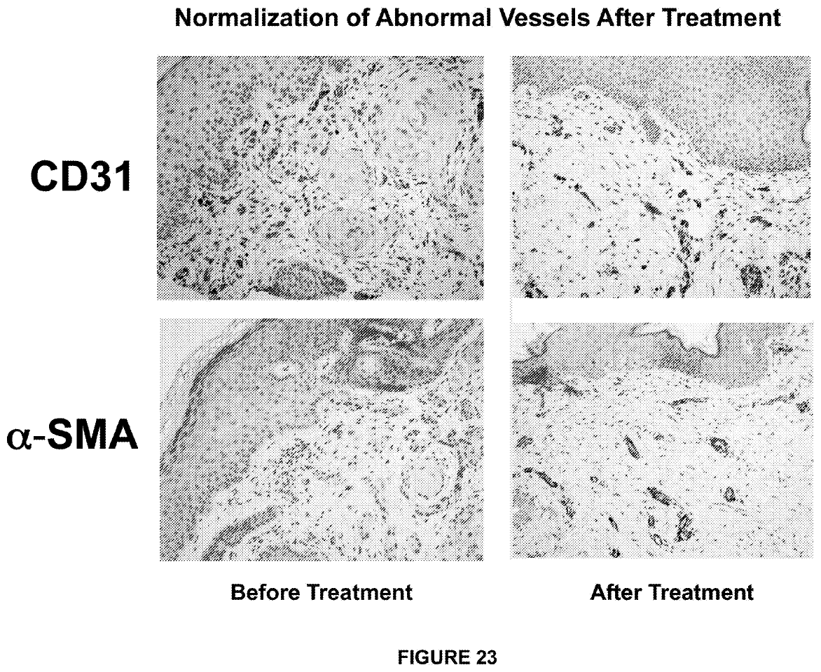

FIG. 23 shows normalization (pruning and maturation) of abnormal vessels after treatment with the topical combinatorial composition. There are decreased tumor blood vessels (CD31 and increased smooth muscle cells (alpha-SMA). The anti-angiogenic effects of the topical combinatorial composition are clearly demonstrated.

### Example 11

### Effect on Quality of Life and Cosmetic Outcome

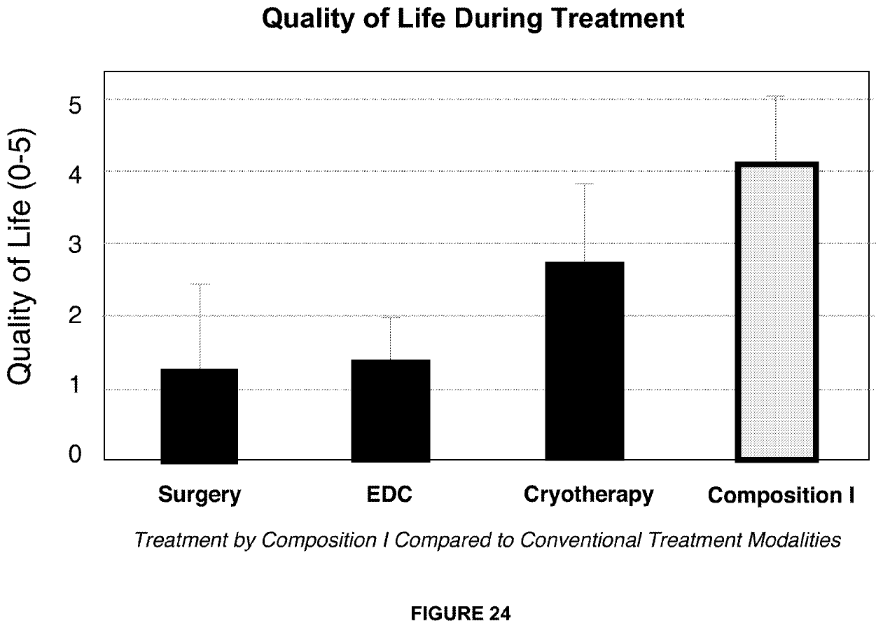

As shown in FIG. 24, the quality of life improved during treatment with Composition I. Patients were queried about their quality of life while on Composition I compared to previous therapies (“5”=no impairment, “0”=major impairment compromising work or normal activities). Treatment with the topical combinatorial composition exhibited the highest quality of life compared to conventional treatments such as surgery, electrodessication and curettage, and cryotherapy.

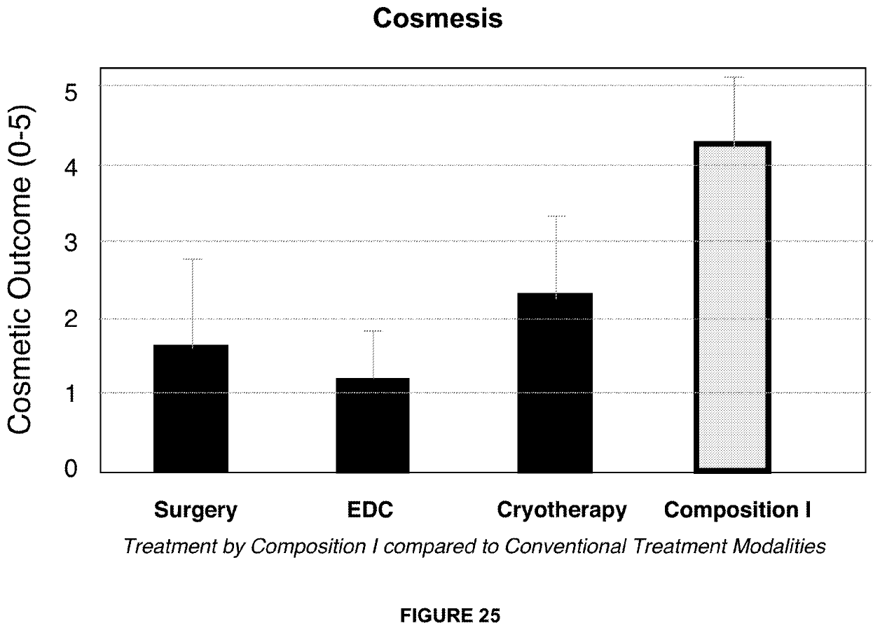

FIG. 25 shows the cosmetic outcomes with treatment of Composition I. Treatment with the topical combinatorial composition exhibited the best cosmetic outcome relative to other treatments. Patients were queried about their cosmetic outcome after successful therapy completion compared to previous treatments (“5”=scarless, “0”=disfiguring scar). Excellent cosmetic outcome was achieved without contour irregularity, atrophy, hypertrophic scar, or depigmentation.

The results fully demonstrate that the topical combinatorial composition improved the quality of life associated with treatment of skin cancer. The results also fully demonstrate that the topical combinatorial composition was rated by patients as superior to conventional treatments in terms of quality of life and cosmesis.

### Example 12

### Treating Oral Squamous Cell Carcinoma

An antiangiogenic regimen was used for treating oral squamous cell carcinoma in non-human vertebrates, for example a dolphin or a dog, based on the multi-targeting combinatorial composition. The regimen included the composition described in Example 2 and 4. The treatment was formulated with a mucoadhesive oral paste base consisting of one or a combination of the following: poly-2-hydroxyethylmathacrylate, carboxymethycellulose, pectin, gelatin.

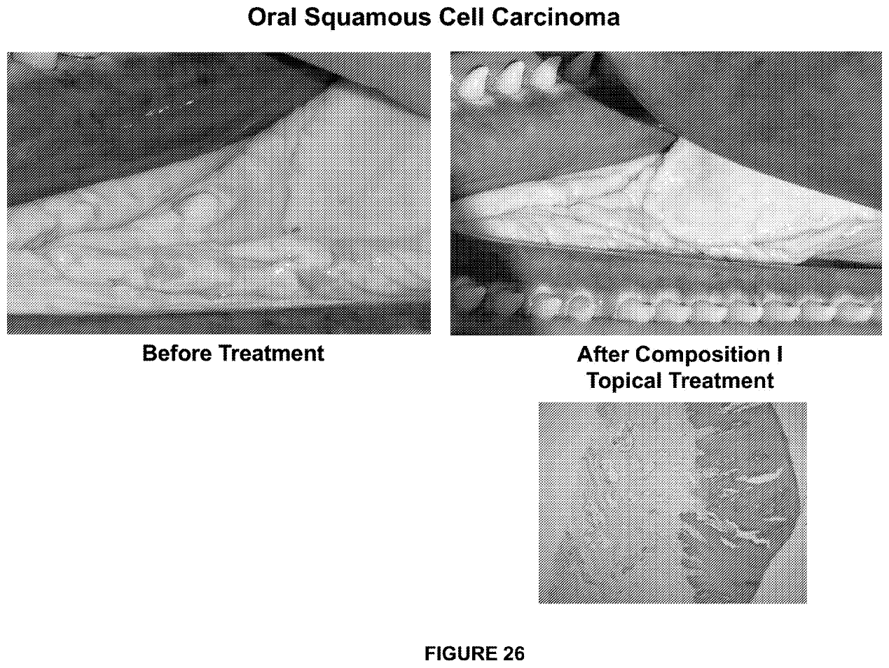

Composition I and Composition II and Composition III are effective in treating oral squamous cell carcinoma. FIG. 26 shows an example case of a oral SCC that was resistant to conventional therapy including surgery, cryotherapy, and radiation. Top left shows before treatment. Top right shows after treatment. Bottom picture shows post-treatment biopsy. The comment on the histopathological assessment of the biopsy after treatment in the dolphin: “degree of improvement in this case was dramatic.” In addition, it was noted that there was overall preservation of the basal layer integrity.

The results demonstrate that the composition of the invention is effective in treating oral squamous cell carcinoma.

### Example 13

### Treating Angiosarcoma

An antiangiogenic regimen was used for treating angiosarcoma based on the combinatorial composition. The regimen included the composition described in Example 1.

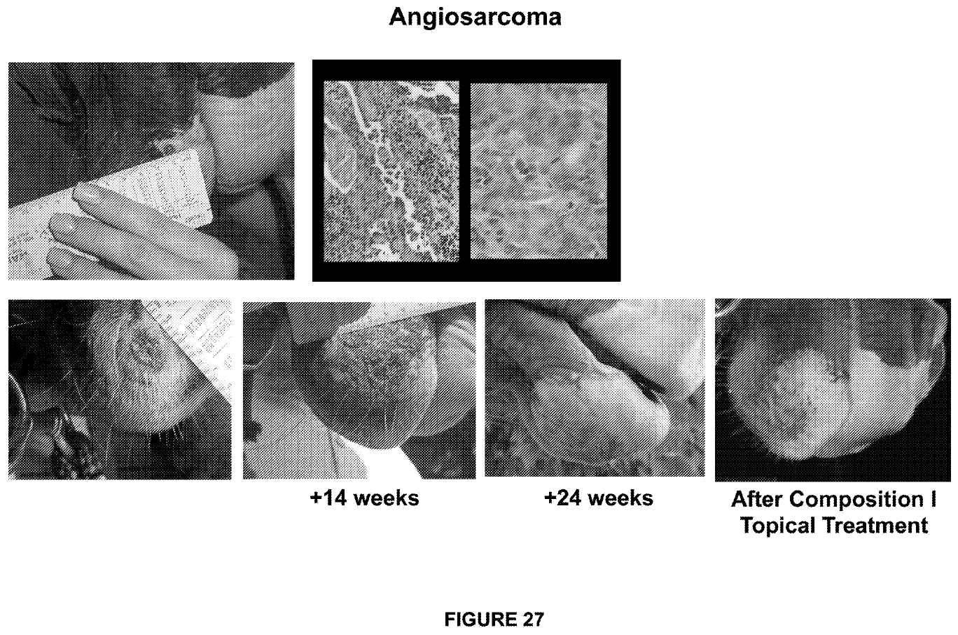

As shown in FIG. 27, Composition I is effective in treating angiosarcoma. The tumor was successfully cleared after 14 weeks of treatment and normalization of skin cosmesis, texture, and pigmentation was achieved within 24 weeks of starting treatment.

### Example 14

### Comparative Reference on Monotherapy Efficacy

Imiquimod monotherapy has been used to treat superficial Basal Cell Carcinoma but is inferior in efficacy to the topical combinatorial composition. Used as monotherapy, the clearance rate (efficacy) is lower, recurrence rate is higher, and adverse event profile more pronounced than that of the topical combinatorial composition. Of note, surprisingly the concentration of imiquimod in the topical combinatorial composition is significantly lower than the concentration used as monotherapy.

Study A

Composite Results of sBCC Clearance:

*Clinical Trials—Efficacy: two double-blind, vehicle-controlled studies (N-364)
Imiquimod 5% cream*—75% (treatment frequency 5×/week×6 weeks)
Vehicle (control)*—2%
Composition I—100%

Study B

Recurrence rates of BCC treated with monotherapy vs topical combinatorial composition is shown below.

| | Imiquimod 5% | |
|---|---|---|
| | Monotherapy | Composition I |
| Time | Recurrence Rate* | Recurrence Rate |
| Post-treatment | 0% | 0% |
| Month 0 | 10% | 0% |
| Month 3 | 13% | 0% |
| Month 6 | 15% | 0% |
| Month 12 | 16% | 0% |
| Month 24 | 21% | 0% |

*Imiquimod Open-Label Clinical Study - Recurrence (N = 162)

### Example 15

### Comparative Reference on Undesirable Local Tissue Reaction

This invention delivers therapeutic outcomes at surprisingly low, otherwise subtherapeutic concentrations. In addition, when the individual agents are used at their normal concentrations and at their standard dose frequencies, the rate of undesirable local reactions is significant. Such undesirable local reactions include itching, burning, bleeding, stinging, pain, tenderness, irritation. For example, in the imiquimod 5% monotherapy clinical studies, the undesirable local reaction (as defined as combined rates of itching, burning, bleeding, stinging, pain/soreness, tenderness, irritation) are shown below:

| | Undesirable Local | |
|---|---|---|
| | Reactions with | Undesirable Local |
| | Imiquimod 5% | Reactions with |
| Clinical Study | monotherapy | Composition I or II |
| 2x/week study | 39% | 0% |
| 3x/week study | 63% | 0% |
| 5x/week study | 28% | 0% |

For other agents in the combinatorial composition, clinical studies of their used as monotherapy at their normal concentration and standard dose frequencies also show a significant rate of undesirable local reaction (as defined as combined rates of stinging, burning, itching, or irritation/dermatitis/rash) are shown below:

| Agent | Undesirable Local Reactions |
|---|---|
| Composition I | 0% |
| Hydrocortisone valerate 0.2% | 7% |
| Tretinoin 0.1% | 15% |
| Calcipotriene 0.005% | 13-45% |

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A composition comprising: a combination of a toll-like receptor 7 (TLR7) agonist, non-steroidal anti-inflammatory drug(s) (NSAIDs), a glucocorticoid anti-inflammatory agent, a vitamin A derivative, a vitamin D3 derivative, a mechanistic target of rapamycin (mTOR) inhibitor or a combination thereof, and a pharmaceutically acceptable carrier.

2. The composition of claim 1, wherein said TLR7 agonist is imiquimod; said NSAID is diclofenac, celecoxib, or a combination thereof; said glucocorticoid anti-inflammatory agent is hydrocortisone valerate; said vitamin A derivative is tretinoin; said vitamin D3 derivative is calcipotriene; or said mTOR inhibitor is sirolimus.

3. The composition of claim 1, wherein said composition comprises imiquimod, diclofenac, hydrocortisone valerate, tretinoin, calcipotriene, celecoxib, sirolimus, or a combination thereof.

4. The composition of claim 3, wherein each ingredient in said combination is present in an amount effective to treat a neoplasia.

5. The composition of claim 3, wherein the combination of agents confers anti-angiogenic and immunotherapeutic activity.

6. The composition of claim 5, wherein imiquimod upregulates endogenous interferon-alpha and interferon-beta and interferon-gamma that downregulates endothelial integrins, inhibits endothelial cell proliferation, migration, and invasion and increases endothelial cell apoptosis.

7. The composition of claim 5, wherein imiquimod upregulates interleukin-12 that decreases production of bFGF and IL-8 that suppress angiogenesis and increases interferon-gamma via T cells and NK cells that modulate the immune response.

8. The composition of claim 5, wherein imiquimod upregulates interleukin-18 that suppresses angiogenesis.

9. The composition of claim 3, wherein the combinations of agents confers antiangiogenic and immunotherapeutic response.

10. The composition of claim 9, wherein celecoxib inhibits Cox-2 which confers antiangiogenic response through inhibition of VEGF and is implicated in conferring resistance to immune detection by cancers.

11. The composition of claim 9, wherein sirolimus confers antiangiogenic response through suppression of an angiogenesis pathway, wherein said pathway is VEGF pathway, and fosters cancer immunotherapy by modulating T regulatory cells and dendritic cells.

12. The composition of claim 9, wherein imiquimod activates the innate immune system through peritumoral and intratumoral infiltration by macrophages and neutrophils which subsequently results in T cell activation.

13. The composition of claim 3, wherein the concentration of one or more drugs are lower than their corresponding therapeutically effective monotherapy concentrations, and wherein said subtherapeutic concentrations are effective when said drugs are present in said combination.

14. The composition of claim 3, wherein said imiquimod is present at the concentration ranging from about 0.1% (w/w) to about 5% (w/w); said calcipotriene is present at the concentration ranging from about 0.0001% (w/w) to about 0.005% (w/w); said tretinoin is present at the concentration ranging from about 0.005% (w/w) to about 0.1% (w/w); said diclofenac is present at the concentration ranging from about 0.1% (w/w) to about 3% (w/w); said hydrocortisone valerate is present at the concentration ranging from about 0.01% (w/w) to about 0.25% (w/w); said celecoxib is present at the concentration ranging from about 1% (w/w) to about 10% (w/w); or said sirolimus is present at the concentration ranging from about 0.01% (w/w) to about 1% (w/w).

15. The composition of claim 1, wherein said composition is a topical composition.

16. The composition of claim 15, wherein said topical composition comprises one or more agents for oral administration.

17. The composition of claim 1, wherein said celecoxib is formulated into a solid dispersion using hydroxypropyl-beta-cyclodextrin.

18. The composition of claim 1, wherein said composition further comprises a skin penetration enhancer that facilitates transcutaneous penetration of ingredients in said composition.

19. The composition of claim 18, wherein said skin penetration enhancer is hyaluronate sodium.

20. The composition of claim 18, wherein said skin penetration enhancer is hydroxypropyl-beta-cyclodextrin and/or hyaluronate.

21. The composition of claim 18, wherein said skin penetration enhancer is hyaluronate, βcyclodextrin, carboxymethylcellulose, pectin, gelatin, or a combination thereof, wherein said hyaluronate is present at the concentration ranging from about 0.0033% (w/w) to about 2.5% (w/w); said βcyclodextrin is present at the concentration ranging from about 5% (w/w) to about 10% (w/w); said carboxymethylcellulose is present at the concentration ranging from about 10% (w/w) to about 35% (w/w); said pectin is present at the concentration ranging from about 1% (w/w) to about 5% (w/w); or said gelatin is present at the concentration ranging from about 2% (w/w) to about 10% (w/w).

22. The composition of claim 1, wherein said composition is in the form of a liquid, a gel, a lotion, a cream, an ointment, a foam, a paste, a powder, a complex solid dispersion, a semisolid structure, an aerosol, or a transdermal delivery vehicle, or a semisolid complex topical dispersion.

23. The composition of claim 1, wherein said composition can be made into an oral mucosa-safe formulation for oral application that utilizes carboxymethylcellulose, pectin, and gelatin or poly-2-hydroxyethylmathacrylate.

24. The composition of claim 1, wherein said composition is capable of targeting angiogenesis to treat a neoplasia.

25. The composition of claim 1, wherein said composition is capable of targeting a skin related organ, an epithelial-related organ, a mouth related organ, a digestive tract related organ; a urinary tract related organ, a reproductive part related organ; a respiratory tract related organ; a gastrointestinal tract related organ, a colorectal tract related organ, an anus related organ, or a tissue surfaces exposed during surgery.

26. The composition of claim 1, wherein said composition is capable of treating a neoplasia.

27. The composition of claim 26, wherein said neoplasia is present in adrenal gland, anus, auditory nerve, bile duct, bladder, bone, brain, breast, central nervous system, cervix, colon, ear, endometrium, esophagus, eye, eyelids, fallopian tube, gastrointestinal tract, head and neck, heart, kidney, larynx, liver, lung, mandible, mandibular condyle, maxilla, mouth, nasopharynx, nose, oral cavity, ovary, pancreas, parotid gland, penis, pinna, pituitary, prostate gland, rectum, retina, salivary gland, skin, small intestine, spinal cord, stomach, testes, thyroid, tonsil, urethra, uterus, vagina, vestibulocochlear nerve and vulva neoplasms, lymph, or lymph node.

28. The composition of claim 26, wherein said neoplasia is a solid tumor.

29. The composition of claim 26, wherein said neoplasia is not a solid tumor.

30. The composition of claim 26, wherein said neoplasia is associated with a lesion.

31. The composition of claim 30, wherein said lesion is a pre-malignant lesion.

32. The composition of claim 30, wherein said lesion is a normal tissue at a risk of transforming into malignancy.

33. The composition of claim 30, wherein said lesion is in a tissue in the setting of immune evasion or immunosuppression.

34. The composition of claim 30, wherein said lesion is hidden or undetected lesion, and wherein said composition is capable of facilitating the detection of said hidden or undetected lesion by revealing the lesion through a mild tissue response that can be detected visually.

35. The composition of claim 30, wherein said lesion is malignant.

36. The composition of claim 30, wherein said lesion is a malignant skin cancer.

37. The composition of claim 30, wherein said lesion is a non-melanoma skin cancer (NMSC).

38. The composition of claim 30, wherein said lesion is a melanoma skin cancer.

39. The composition of claim 37, wherein said NMSC is a basal cell carcinoma (BCC), a squamous cell carcinoma in situ (SCCIS), a squamous cell carcinoma (SCC), an angiosarcoma, a cutaneous B-cell lymphoma, a cutaneous T-cell lymphoma, a dermatofibrosarcoma, a dermatofibrosarcoma protuberans, a Merkel cell carcinoma, or a sebaceous carcinoma.

40. The composition of claim 38, wherein said melanoma skin cancer is lentigo maligna melanoma, superficial spreading melanoma, acral lentiginous melanoma, mucosal melanoma, nodular melanoma, polypoid melanoma, desmoplastic melanoma, small-cell melanoma, spitzoid melanoma, uveal melanoma, amelanotic melanoma, or nevoid melanoma.

41. A method of treating a neoplasia in a subject, the method comprising: administering to said subject a therapeutically effective amount of a toll-like receptor 7 (TLR7) agonist, a non-steroidal anti-inflammatory drug (NSAID), a glucocorticoid anti-inflammatory agent, a vitamin A derivative, a vitamin D3 derivative, a mechanistic target of rapamycin (mTOR) inhibitor, or a combination thereof, thereby treating said neoplasia in said subject.

42. The method of claim 41, wherein said TLR7 agonist is imiquimod; said NSAID is diclofenac, celecoxib, or a combination thereof; said glucocorticoid anti-inflammatory agent is hydrocortisone valerate; said vitamin A derivative is tretinoin; said vitamin D3 derivative is calcipotriene; or said mTOR inhibitor is sirolimus.

43. The method of claim 42, wherein the administration comprises administering imiquimod, diclofenac, hydrocortisone valerate, tretinoin, calcipotriene, celecoxib, sirolimus, or a combination thereof.

44. The method of claim 42, wherein the administration comprises administering a composition comprising imiquimod, diclofenac, hydrocortisone valerate, tretinoin, calcipotriene, celecoxib, sirolimus, or a combination thereof, and wherein each ingredient in said combination is present in an amount effective to treat a neoplasia.

45. The method of claim 42, wherein said imiquimod is present at the concentration ranging from about 0.3% (w/w) to about 5% (w/w); said calcipotriene is present at the concentration ranging from about 0.0001% (w/w) to about 0.005% (w/w); said tretinoin is present at the concentration ranging from about 0.005% (w/w) to about 0.1% (w/w); said diclofenac is present at the concentration ranging from about 0.1% (w/w) to about 3% (w/w); said hydrocortisone valerate is present at the concentration ranging from about 0.01% (w/w) to about 0.25% (w/w); said celecoxib is present at the concentration ranging from about 1% (w/w) to about 10% (w/w); or said sirolimus is present at the concentration ranging from about 0.01% (w/w) to about 1% (w/w).

46. The method of claim 41, wherein the step of administering is performed topically.

47. The method of claim 44, wherein said composition further comprises a skin penetration enhancer that facilitates transcutaneous penetration of ingredients in said composition.

48. The method of claim 47, wherein said skin penetration enhancer is hyaluronate sodium or β-cyclodextrin.

49. The method of claim 47, wherein said skin penetration enhancer is hyaluronate, βcyclodextrin, carboxymethylcellulose, pectin, gelatin, or a combination thereof, wherein said hyaluronate is present at the concentration ranging from about 0.0033% (w/w) to about 2.5% (w/w); said βcyclodextrin is present at the concentration ranging from about 5% (w/w) to about 10% (w/w); said carboxymethylcellulose is present at the concentration ranging from about 10% (w/w) to about 35% (w/w); said pectin is present at the concentration ranging from about 1% (w/w) to about 5% (w/w); or said gelatin is present at the concentration ranging from about 2% (w/w) to about 10% (w/w).

50. The method of claim 44, wherein said composition is in the form of a liquid, a gel, a lotion, a cream, an ointment, a foam, a paste, a powder, a semisolid structure, an aerosol, or a transdermal delivery vehicle.

51. The method of claim 41, wherein said neoplasia is present in adrenal gland, anus, auditory nerve, bile duct, bladder, bone, brain, breast, central nervous system, cervix, colon, ear, endometrium, esophagus, eye, eyelids, fallopian tube, gastrointestinal tract, head and neck, heart, kidney, larynx, liver, lung, mandible, mandibular condyle, maxilla, mouth, nasopharynx, nose, oral cavity, ovary, pancreas, parotid gland, penis, pinna, pituitary, prostate gland, rectum, retina, salivary gland, skin, small intestine, spinal cord, stomach, testes, thyroid, tonsil, urethra, uterus, vagina, vestibulocochlear nerve and vulva neoplasms, lymph, or lymph node.

52. The method of claim 41, wherein said neoplasia is a solid tumor.

53. The method of claim 41, wherein said neoplasia is not a solid tumor.

54. The method of claim 41, wherein said neoplasia is associated with a lesion.

55. The method of claim 54, wherein said lesion is a pre-malignant lesion.

56. The method of claim 54, wherein said lesion is a normal tissue at a risk of transforming into malignancy.

57. The method of claim 54, wherein said lesion is in a tissue in the setting of immune evasion or immunosuppression.

58. The method of claim 54, wherein said lesion is hidden or undetected lesion, and wherein said administration facilitates the detection of said hidden or undetected lesion.

59. The method of claim 54, wherein said lesion is malignant.

60. The method of claim 54, wherein said lesion is a malignant skin cancer.

61. The method of claim 54, wherein said lesion is a non-melanoma skin cancer (NMSC).

62. The method of claim 61, wherein said NMSC is a basal cell carcinoma (BCC), a squamous cell carcinoma (SCC), a squamous cell carcinoma in situ (SCCIS), an actinic keratosis (AK), an angiosarcoma, a cutaneous B-cell lymphoma, a cutaneous T-cell lymphoma, a dermatofibrosarcoma, a dermatofibrosarcoma protuberans, a Merkel cell carcinoma, or a sebaceous carcinoma.

63. The method of claim 54, wherein said lesion is a melanoma skin cancer.

64. The method of claim 63, wherein said melanoma skin cancer is lentigo maligna melanoma, superficial spreading melanoma, acral lentiginous melanoma, mucosal melanoma, nodular melanoma, polypoid melanoma, desmoplastic melanoma, small-cell melanoma, spitzoid melanoma, uveal melanoma, amelanotic melanoma, or nevoid melanoma.

65. The method of claim 41, wherein said neoplasia is a non-cancerous neoplasia.

66. The method of claim 65, wherein said non-cancerous neoplasia is angiomas, epitheliomas, papillomas, adenomas, fibromas, sarcomas, haemangiomas, lipomas, chondromas, osteomas, histiocytomas, leiomyomas, rhabdomyomas, meningiomas, Schwannomas, neurilemmoma, myomas, naevi, neuromas, osteochondromas, benign sinonasals, sebaceous hyperplasia, seborrheic keratosis, and papillomas

67. The method of claim 41, said ingredients are co-administered.

68. The method of claim 41, said ingredients are administered independently.

69. The method of claim 41, further comprising the step of determining a dosage of said ingredients for said subject, prior to the step of administering to said subject.

70. The method of claim 69, wherein said dosage is determined based on an algorithmic method by which therapeutic efficacy can be achieved without undesirable local reaction.

71. The method of claim 70, further comprising the steps of monitoring and guiding a dose frequency through a direct observation, a remote assessment of images and symptoms by a mobile telephony, or an automated assessment; and providing recommendation via computer image analysis and artificial intelligence.

72. A method of targeting angiogenesis to treat a neoplasia in a subject, the method comprising: administering to said subject a therapeutically effective amount of a toll-like receptor 7 (TLR7) agonist, a non-steroidal anti-inflammatory drug (NSAID), a glucocorticoid anti-inflammatory agent, a vitamin A derivative, a vitamin D3 derivative, a mechanistic target of rapamycin (mTOR) inhibitor, or a combination thereof, thereby targeting said angiogenesis to treat said neoplasia in said subject.

73. A method for improving a quality of life during a skin cancer treatment in a subject, the method comprising: administering to said subject a therapeutically effective amount of a toll-like receptor 7 (TLR7) agonist, a non-steroidal anti-inflammatory drug (NSAID), a glucocorticoid anti-inflammatory agent, a vitamin A derivative, a vitamin D3 derivative, or a combination thereof, thereby improving said quality of life during said skin cancer treatment in said subject.

74. A method for improving a cosmetic outcome or cosmesis associated with a skin cancer treatment in a subject, the method comprising: administering to said subject a therapeutically effective amount of a toll-like receptor 7 (TLR7) agonist, a non-steroidal anti-inflammatory drug (NSAID), a glucocorticoid anti-inflammatory agent, a vitamin A derivative, a vitamin D3 derivative, or a combination thereof, thereby improving said cosmetic outcome or cosmesis associated with said skin cancer treatment in said subject.

75. A method for treating an oral squamous cell carcinoma in a subject, the method comprising: administering to said subject a therapeutically effective amount of a toll-like receptor 7 (TLR7) agonist, a non-steroidal anti-inflammatory drug (NSAID), a glucocorticoid anti-inflammatory agent, a vitamin A derivative, a vitamin D3 derivative, or a combination thereof, thereby treating said oral squamous cell carcinoma in said subject.

76. A method for treating an angiosarcoma in a subject, the method comprising: administering to said subject a therapeutically effective amount of a toll-like receptor 7 (TLR7) agonist, a non-steroidal anti-inflammatory drug (NSAID), a glucocorticoid anti-inflammatory agent, a vitamin A derivative, a vitamin D3 derivative, or a combination thereof, thereby treating said angiosarcoma in said subject.

77. A method that permits treating large surface areas of field cancerization on the skin, the method comprising: administering to a subject the composition of any one of claims 1-40.

78. A method for neoadjuvant treatment of neoplasia in which the intended objective is to improve the outcome of different treatment administered or performed sequentially and/or to diminish one or more undesirable consequences of the other treatment, the method comprising: administering to a subject the composition of any one of claims 1-40.

79. The method of claim 78, wherein said different treatment is a surgery.

80. The method of claim 78, wherein at least one of said undesirable consequences is a scar.

81. A method to treat immunosuppressed patients who are at markedly elevated risk of developed skin neoplasms, the method comprising: administering to a subject the composition of any one of claims 1-40.

82. A method to dispense a specific amount of topical therapy in a metered-dose-fashion, the method comprising: administering to a subject the composition of any one of claims 1-40.
